(19) United States
(12) Patent Application Publication
(10) Pub. No.: US 2026/0007331 A1
(43) Pub. Date: Jan. 8, 2026

(54) METHODS AND DEVICES FOR DETERMINING BODY POSE USING FIBER OPTIC COMPONENTS

(71) Applicant: Meta Platforms Technologies, LLC, Menlo Park, CA (US)

(72) Inventors: Ergys Ristani, Bellevue, WA (US); Burkley Delesdernier Patterson, Catonsville, MD (US); Sean Braxton, Redmond, WA (US); Francesco Marsili, Foster City, CA (US); Taylor Douglas Niehues, Snohomish, WA (US); Michael Hurst, Seattle, WA (US); Li Guan, Bellevue, WA (US)

(21) Appl. No.: 19/260,166

(22) Filed: Jul. 3, 2025

Related U.S. Application Data
(60) Provisional application No. 63/667,688, filed on Jul. 3, 2024.

(51) Int. Cl. A61B 5/11; A61B 5/00; G01D 5/353; G06F 3/01; G06F 3/03; G06F 3/0346

(52) U.S. Cl. CPC A61B 5/1126; A61B 5/1116; A61B 5/6806; G01D 5/35316; G06F 3/0304; G06F 3/0346; G06F 3/014

(57) ABSTRACT

An example device includes a wearable structure configured to couple to a user's body. The wearable structure includes a light emitter coupled to a plurality of fiber optic components and configured to transmit a light output and an optical switch coupled to the plurality of fiber optic components. The wearable structure further includes a photo sensor coupled to the optical switch. The photo sensor is configured to detect a reflected portion of the light output from the light emitter and output a corresponding photo sensor signal. The wearable structure also includes control circuitry coupled to the photo sensor and configured to determine a curvature of a respective fiber optic component based on analysis of the corresponding photo sensor signal, and determine a pose of the user based on the curvature of the respective fiber optic component.

### RELATED APPLICATION

This application claims priority to U.S. Provisional Application Ser. No. 63/667,688, filed Jul. 3, 2024, entitled “Detecting Arm, Hand and/or Finger Pose With A Compact Wearable Device, And Systems And Methods Of Use Thereof,” which is incorporated herein by reference.

### TECHNICAL FIELD

This relates generally to wearable technology devices for pose estimation and movement analysis including, but not limited to, wearable devices having fiber optic components for use with pose estimation.

### BACKGROUND

Wearable technology has gained traction across various industries, including healthcare, fitness, and entertainment. The wearable devices offer users the ability to monitor physiological parameters, track physical activity, and interact with digital environments. As the demand for more sophisticated and accurate wearable devices increases, there is a growing need for innovative solutions that enhance user experience and provide precise data.

Some wearable devices rely on electronic sensors to detect movement and gather data. However, the sensors being used have limitations and drawbacks. Cameras, for example, suffer from occlusion issues due to their reliance on having a clear line of sight, making them unsuitable for applications where unobstructed views cannot be guaranteed. Inertial Measurement Units (IMUs) and similar sensors, while useful, are prone to drift over time, leading to inaccuracies in long-term measurements.

As such, there is a need to address one or more of the above-identified challenges. A brief summary of solutions to the issues noted above are described below.

### SUMMARY

Fiber optic technology presents a promising avenue for advancing wearable devices. Unlike IMUs, fiber optics do not suffer from drift, providing consistent and reliable data over extended periods. Additionally, fiber optics are non-magnetic and immune to electromagnetic interference, which can affect other non-line-of-sight sensors when exposed to metal or other signals.

Other bend sensors lack the high angular resolution that fiber optics offer, making fiber optics superior for applications requiring precise motion tracking. By utilizing light transmission and reflection, fiber optic components can provide high-resolution data on movement and positioning. This technology is particularly advantageous due to its lightweight nature, flexibility, and immunity to electromagnetic interference.

The integration of fiber optic components into wearable devices allows for the continuous monitoring of user movements with minimal intrusion. By analyzing the curvature of fiber optic components, it is possible to determine the pose of the user accurately. This capability is important for applications that require precise motion tracking, such as virtual reality, physical rehabilitation, and sports performance analysis.

Despite the potential benefits, the implementation of fiber optic technology in wearable devices poses several technical challenges. These include the need for efficient light transmission, accurate detection of reflected light, and the development of control systems capable of processing complex data in real-time.

The present invention addresses these challenges by providing a device that incorporates a wearable structure with integrated fiber optic components. This device is designed to determine user pose through the analysis of light reflection and curvature, offering a novel approach to enhancing the functionality and accuracy of wearable technology.

An example device, including a plurality of fiber optic components and a wearable structure configured to couple to the body of a user, is described herein. This example wearable structure includes a light emitter coupled to the plurality of fiber optic components and configured to transmit a light output to the plurality of fiber optic components. The wearable structure further includes an optical switch coupled to the plurality of fiber optic components and having a plurality of inputs and one or more outputs and a photo sensor coupled to at least one output of the one or more outputs of the optical switch. The photo sensor is configured to detect a reflected portion of the light output from the light emitter and output a corresponding photo sensor signal. The wearable structure further includes control circuitry coupled to the photo sensor. The control circuitry is configured to (i) determine a curvature of a respective fiber optic component of the plurality of fiber optic components based on analysis of the corresponding photo sensor signal and (ii) determine a pose of the user based on the curvature of the respective fiber optic component. The wearable structure further includes a power source configured to provide power to the control circuitry, the light emitter, the optical switch, and the photo sensor. Each fiber optic component of the plurality of fiber optic components includes a respective set of passive sensors. Each passive sensor of the respective set of passive sensors is configured to reflect a portion of light transmitted to the fiber optic component.

The devices and/or systems described herein can be configured to include instructions that cause the performance of methods and operations associated with the presentation and/or interaction with an extended-reality (XR) headset. These methods and operations can be stored on a non-transitory computer-readable storage medium of a device or a system. It is also noted that the devices and systems described herein can be part of a larger, overarching system that includes multiple devices. A non-exhaustive of list of electronic devices that can, either alone or in combination (e.g., a system), include instructions that cause the performance of methods and operations associated with the presentation and/or interaction with an XR experience include an extended-reality headset (e.g., a mixed-reality (MR) headset or a pair of augmented-reality (AR) glasses as two examples), a wrist-wearable device, an intermediary processing device, a smart textile-based garment, etc. For example, when an XR headset is described, it is understood that the XR headset can be in communication with one or more other devices (e.g., a wrist-wearable device, server, or an intermediary processing device) which together can include instructions for performing methods and operations associated with the presentation and/or interaction with an extended-reality system (i.e., the XR headset would be part of a system that includes one or more additional devices). Multiple combinations with different related devices are envisioned, but not recited for brevity.

Instructions that cause performance of the methods and operations described herein can be stored on a non-transitory computer readable storage medium. The non-transitory computer-readable storage medium can be included on a single electronic device or spread across multiple electronic devices of a system (computing system). A non-exhaustive of list of electronic devices that can either alone or in combination (e.g., a system) perform the method and operations described herein include XR headset/glasses (e.g., a mixed-reality (MR) headset or a pair of augmented-reality (AR) glasses as two examples), a wrist-wearable device, an intermediary processing device, a smart textile-based garment, etc. For instance, the instructions can be stored on a pair of AR glasses or can be stored on a combination of a pair of AR glasses and an associated input device (e.g., a wrist-wearable device) such that instructions for causing detection of input operations can be performed at the input device and instructions for causing changes to a displayed user interface in response to those input operations can be performed at the pair of AR glasses. The devices and systems described herein can be configured to be used in conjunction with methods and operations for providing an XR experience. The methods and operations for providing an XR experience can be stored on a non-transitory computer-readable storage medium.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIGS. 1A-1B illustrate an example wearable device with fiber optic components configured to measure and determine a user's movements, in accordance with some embodiments.

FIG. 2 illustrates an example wearable device including one or more fiber optic components, in accordance with some embodiments.

FIGS. 3A and 3B illustrate example wearable devices including one or more fiber optic components, in accordance with some embodiments.

FIGS. 4A-4C illustrate the fiber Bragg grating principles and example configurations of the fiber optic components, in accordance with some embodiments.

FIG. 5 shows an example method flow chart for pose estimation, in accordance with some embodiments.

FIGS. 6A, 6B, 6C-1, and 6C-2 illustrate example MR and AR systems, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

As an illustrative example, a wearable device (e.g., a head-wearable device, a wrist-wearable device, or other type of wearable device) may be configured to determine a curvature of respective fiber optic components based on analysis of corresponding reflected signals. The wearable device may be further configured to determine a pose of a user based on the curvature of the respective fiber optic components. Using fiber optic components to determine curvature and pose avoids the obstruction issues that line-of-sight sensors face. Additionally, fiber-optic based pose measurements are less susceptible to electromagnet interference and drift issues encounter with other types of sensors (e.g., IMU sensors).

### Overview

Embodiments of this disclosure can include or be implemented in conjunction with various types of extended-realities (XRs) such as mixed-reality (MR) and augmented-reality (AR) systems. MRs and ARs, as described herein, are any superimposed functionality and/or sensory-detectable presentation provided by MR and AR systems within a user's physical surroundings. Such MRs can include and/or represent virtual realities (VRs) and VRs in which at least some aspects of the surrounding environment are reconstructed within the virtual environment (e.g., displaying virtual reconstructions of physical objects in a physical environment to avoid the user colliding with the physical objects in a surrounding physical environment). In the case of MRs, the surrounding environment that is presented through a display is captured via one or more sensors configured to capture the surrounding environment (e.g., a camera sensor, time-of-flight (ToF) sensor). While a wearer of an MR headset can see the surrounding environment in full detail, they are seeing a reconstruction of the environment reproduced using data from the one or more sensors (i.e., the physical objects are not directly viewed by the user). An MR headset can also forgo displaying reconstructions of objects in the physical environment, thereby providing a user with an entirely VR experience. An AR system, on the other hand, provides an experience in which information is provided, e.g., through the use of a waveguide, in conjunction with the direct viewing of at least some of the surrounding environment through a transparent or semi-transparent waveguide(s) and/or lens(es) of the AR glasses. Throughout this application, the term “extended reality (XR)” is used as a catchall term to cover both ARs and MRs. In addition, this application also uses, at times, a head-wearable device or headset device as a catchall term that covers XR headsets such as AR glasses and MR headsets.

As alluded to above, an MR environment, as described herein, can include, but is not limited to, non-immersive, semi-immersive, and fully immersive VR environments. As also alluded to above, AR environments can include marker-based AR environments, markerless AR environments, location-based AR environments, and projection-based AR environments. The above descriptions are not exhaustive and any other environment that allows for intentional environmental lighting to pass through to the user would fall within the scope of an AR, and any other environment that does not allow for intentional environmental lighting to pass through to the user would fall within the scope of an MR.

The AR and MR content can include video, audio, haptic events, sensory events, or some combination thereof, any of which can be presented in a single channel or in multiple channels (such as stereo video that produces a three-dimensional effect to a viewer). Additionally, AR and MR can also be associated with applications, products, accessories, services, or some combination thereof, which are used, for example, to create content in an AR or MR environment and/or are otherwise used in (e.g., to perform activities in) AR and MR environments.

Interacting with these AR and MR environments described herein can occur using multiple different modalities and the resulting outputs can also occur across multiple different modalities. In one example AR or MR system, a user can perform a swiping in-air hand gesture to cause a song to be skipped by a song-providing application programming interface (API) providing playback at, for example, a home speaker.

A hand gesture, as described herein, can include an in-air gesture, a surface-contact gesture, and or other gestures that can be detected and determined based on movements of a single hand (e.g., a one-handed gesture performed with a user's hand that is detected by one or more sensors of a wearable device (e.g., electromyography (EMG) and/or inertial measurement units (IMUs) of a wrist-wearable device, and/or one or more sensors included in a smart textile wearable device) and/or detected via image data captured by an imaging device of a wearable device (e.g., a camera of a head-wearable device, an external tracking camera setup in the surrounding environment)). “In-air” generally includes gestures in which the user's hand does not contact a surface, object, or portion of an electronic device (e.g., a head-wearable device or other communicatively coupled device, such as the wrist-wearable device), in other words the gesture is performed in open air in 3D space and without contacting a surface, an object, or an electronic device. Surface-contact gestures (contacts at a surface, object, body part of the user, or electronic device) more generally are also contemplated in which a contact (or an intention to contact) is detected at a surface (e.g., a single-or double-finger tap on a table, on a user's hand or another finger, on the user's leg, a couch, a steering wheel). The different hand gestures disclosed herein can be detected using image data and/or sensor data (e.g., neuromuscular signals sensed by one or more biopotential sensors (e.g., EMG sensors) or other types of data from other sensors, such as proximity sensors, ToF sensors, sensors of an IMU, capacitive sensors, strain sensors) detected by a wearable device worn by the user and/or other electronic devices in the user's possession (e.g., smartphones, laptops, imaging devices, intermediary devices, and/or other devices described herein).

The input modalities as alluded to above can be varied and are dependent on a user's experience. For example, in an interaction in which a wrist-wearable device is used, a user can provide inputs using in-air or surface-contact gestures that are detected using neuromuscular signal sensors of the wrist-wearable device. In the event that a wrist-wearable device is not used, alternative and entirely interchangeable input modalities can be used instead, such as camera(s) located on the headset/glasses or elsewhere to detect in-air or surface-contact gestures or inputs at an intermediary processing device (e.g., through physical input components (e.g., buttons and trackpads)). These different input modalities can be interchanged based on both desired user experiences, portability, and/or a feature set of the product (e.g., a low-cost product may not include hand-tracking cameras).

While the inputs are varied, the resulting outputs stemming from the inputs are also varied. For example, an in-air gesture input detected by a camera of a head-wearable device can cause an output to occur at a head-wearable device or control another electronic device different from the head-wearable device. In another example, an input detected using data from a neuromuscular signal sensor can also cause an output to occur at a head-wearable device or control another electronic device different from the head-wearable device. While only a couple examples are described above, one skilled in the art would understand that different input modalities are interchangeable along with different output modalities in response to the inputs.

Specific operations described above may occur as a result of specific hardware. The devices described are not limiting and features on these devices can be removed or additional features can be added to these devices. The different devices can include one or more analogous hardware components. For brevity, analogous devices and components are described herein. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device, a head-wearable device, a handheld intermediary processing device (HIPD), a smart textile-based garment, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., VR animations, such as three-dimensional modeling); (iv) a field-programmable gate array (FPGA) that can be programmed and reconfigured after manufacturing and/or customized to perform specific tasks, such as signal processing, cryptography, and machine learning; or (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronic components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or (iv) DSPs. As described herein, a graphics module is a component or software module that is designed to handle graphical operations and/or processes and can include a hardware module and/or a software module.

As described herein, memory refers to electronic components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or (v) any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronic components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) pogo pins, which may be small, spring-loaded pins configured to provide a charging interface; (v) wireless charging interfaces; (vi) global-positioning system (GPS) interfaces; (vii) Wi-Fi interfaces for providing a connection between a device and a wireless network; and (viii) sensor interfaces.

As described herein, sensors are electronic components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device, such as a simultaneous localization and mapping (SLAM) camera); (ii) biopotential-signal sensors (used interchangeably with neuromuscular-signal sensors); (iii) IMUs for detecting, for example, angular rate, force, magnetic field, and/or changes in acceleration; (iv) heart rate sensors for measuring a user's heart rate; (v) peripheral oxygen saturation (SpO2) sensors for measuring blood oxygen saturation and/or other biometric data of a user; (vi) capacitive sensors for detecting changes in potential at a portion of a user's body (e.g., a sensor-skin interface) and/or the proximity of other devices or objects; (vii) sensors for detecting some inputs (e.g., capacitive and force sensors); and (viii) light sensors (e.g., ToF sensors, infrared light sensors, or visible light sensors), and/or sensors for sensing data from the user or the user's environment. As described herein biopotential-signal-sensing components are devices used to measure electrical activity within the body (e.g., biopotential-signal sensors). Some types of biopotential-signal sensors include (i) electroencephalography (EEG) sensors configured to measure electrical activity in the brain to diagnose neurological disorders; (ii) electrocardiogramar EKG) sensors configured to measure electrical activity of the heart to diagnose heart problems; (iii) EMG sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications; (x) camera applications; (xi) web-based applications; (xii) health applications; (xiii) AR and MR applications; and/or (xiv) any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). A communication interface can refer to a software layer that enables different software programs to communicate with each other (e.g., APIs and protocols such as HTTP and TCP/IP).

As described herein, a graphics module is a component or software module that is designed to handle graphical operations and/or processes and can include a hardware module and/or a software module.

As described herein, non-transitory computer-readable storage media are physical devices or storage medium that can be used to store electronic data in a non-transitory form (e.g., such that the data is stored permanently until it is intentionally deleted and/or modified).

### Pose Estimation

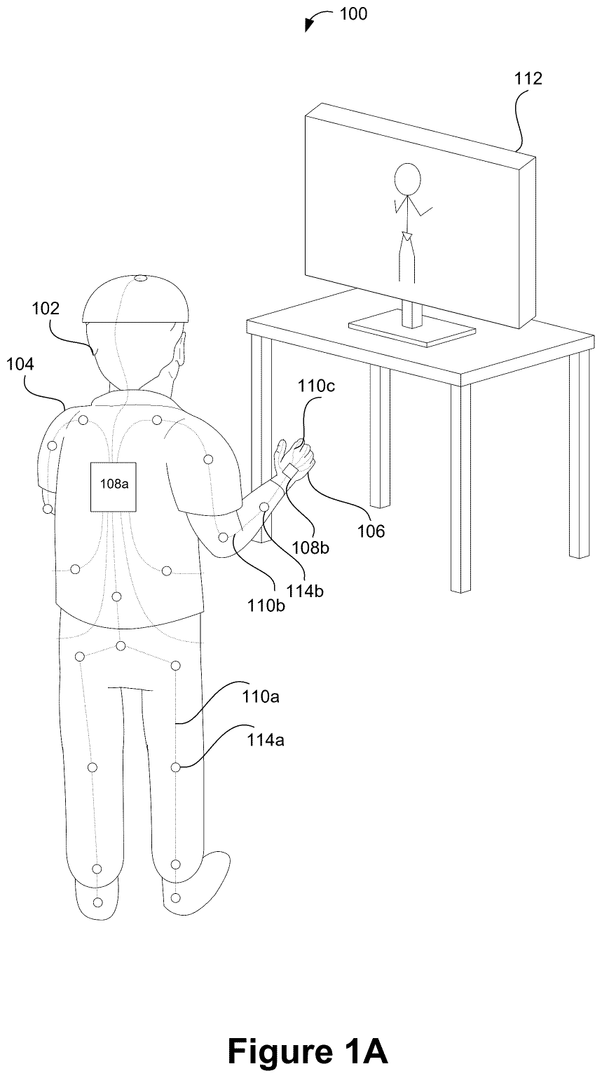

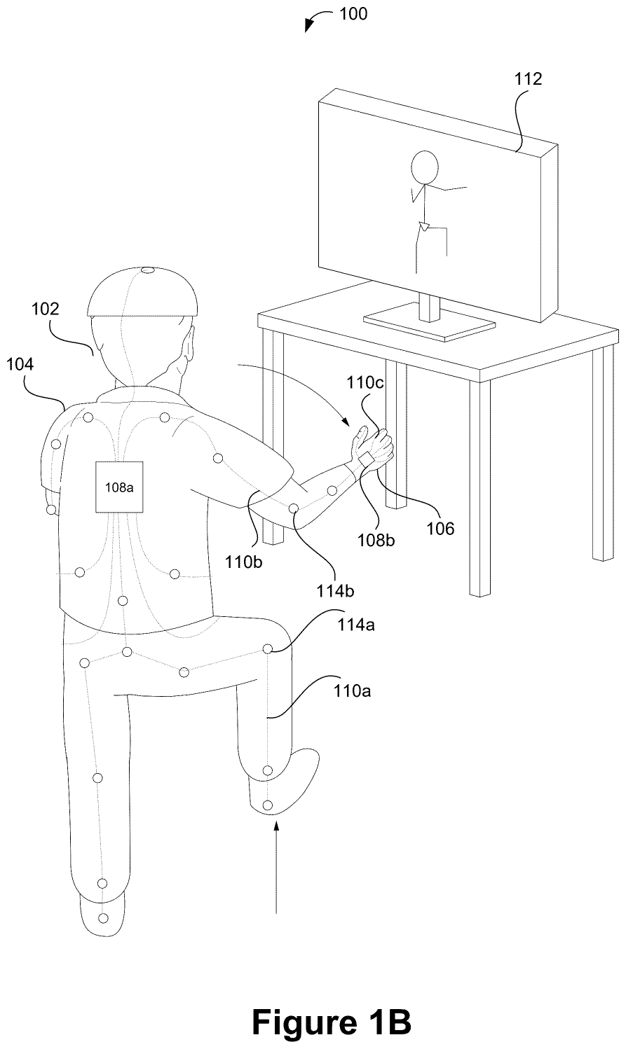

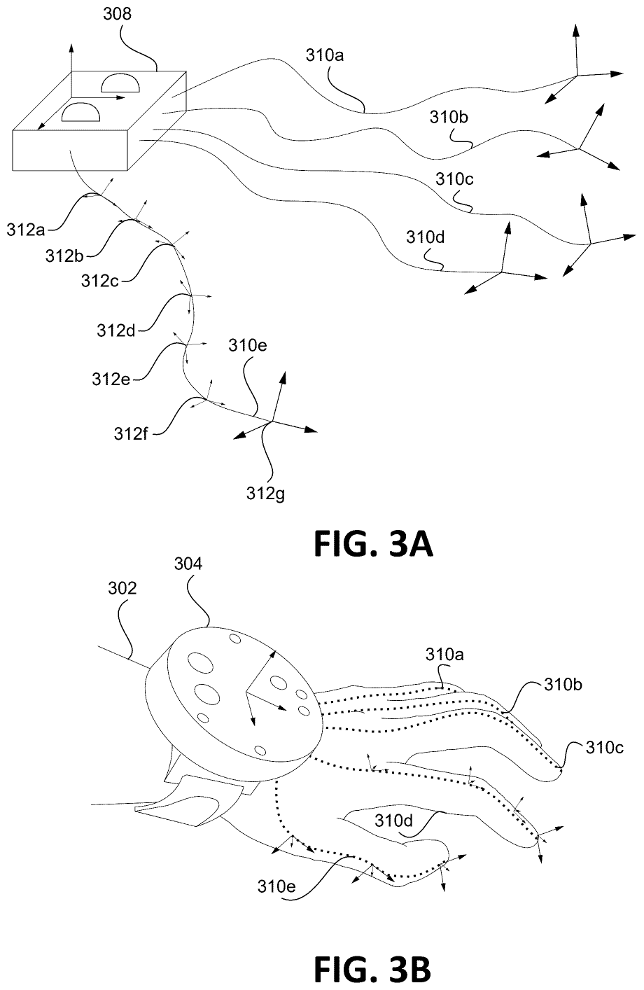

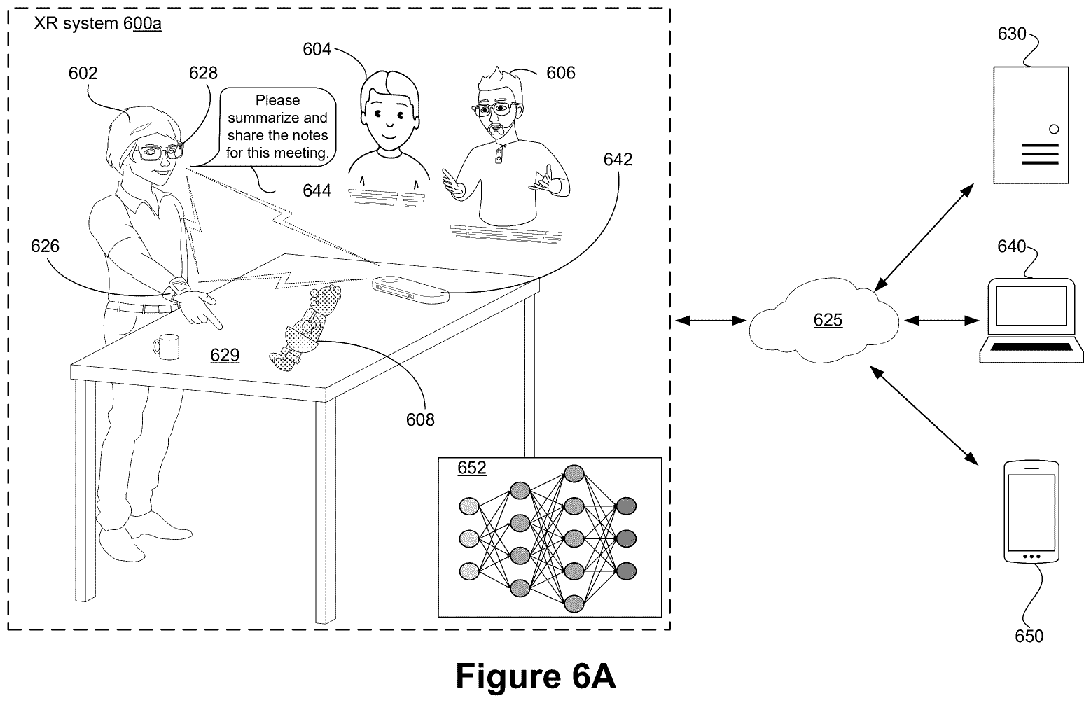

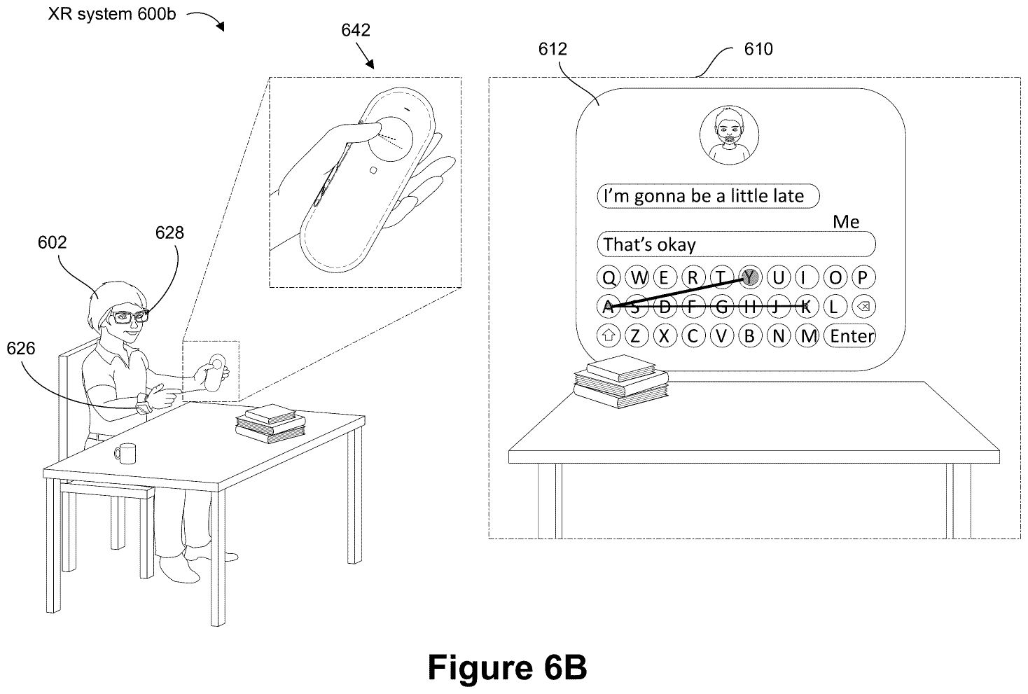

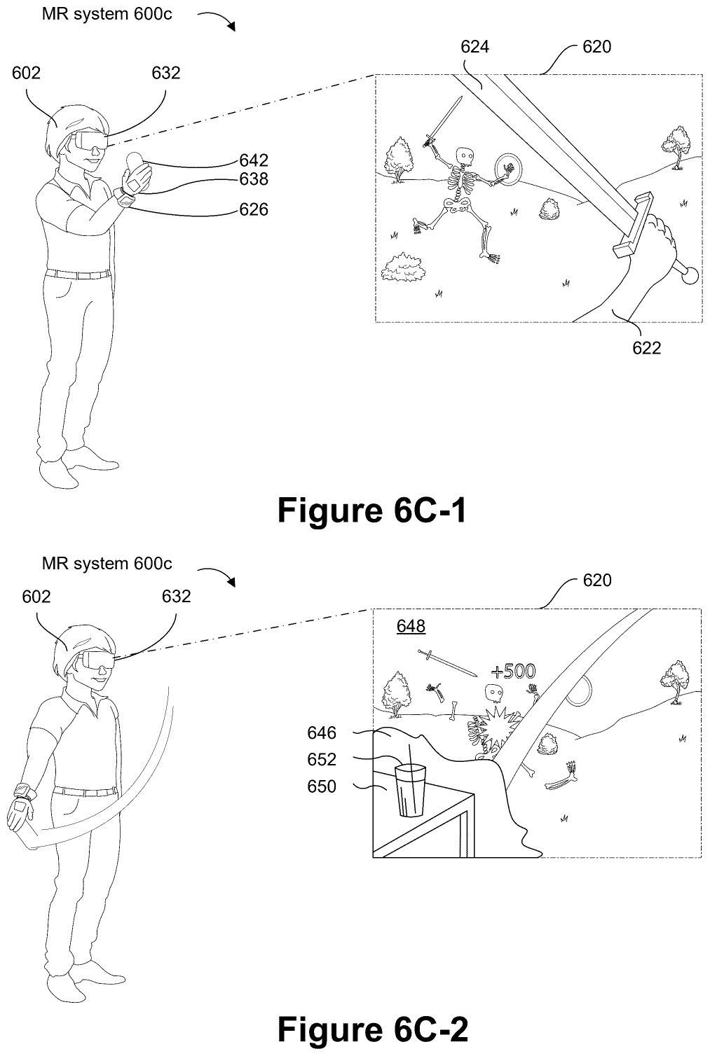

FIGS. 1A-1B illustrate an example wearable device with fiber optic components configured to measure and determine a user's movements, in accordance with some embodiments. The example wearable device includes a wearable structure configured to couple and conform to the body of a user. The wearable structure may be further configured to track hand, fingertip, and body pose with high accuracy using a compact wearable sensor. The wearable device may include at least one of a hand-wearable device 106 (e.g., a glove or smart textile-based garments 638; FIG. 6C-1), a wrist-wearable device (e.g., wrist-wearable device 304; FIG. 3B), and/or a body-wearable device 104 (e.g., a body suit) that can be worn comfortably on various parts of the body, such as the wrist or arm. The wearable structure may serve as the foundation for integrating the device's components, ensuring both functionality and user comfort.

Turning to FIG. 1A, a scene 100 illustrates a user 102 at a first point in time. The user 102 is wearing a body-wearable device 104 while the user 102 is performing movements. In the example of FIG. 1A, the user 102 is also wearing a hand-wearable device 106. The scene 100 further shows the user's movements replicated on a screen 112. In some embodiments, the screen 112 is virtual screen projected via the display of an augmented-reality (AR) headset, AR, smart glasses, and/or a virtual reality (VR) headset.

Each wearable device in FIG. 1A includes a respective compute core. The compute core may include a housing with one or more circuit components (e.g., a light emitter, a light sensor, and control circuitry). The body-wearable device 104 includes a compute core 108a and the hand-wearable device 106 includes a compute core 108b, collectively referred to as the compute cores 108. In some embodiments, one compute core is coupled to multiple wearable devices (e.g., multiple wearable devices share a same compute core). In some embodiments, a compute core is coupled to at least one of the hand-wearable device 106, the portion of the body-wearable device 104 corresponding to the user's back, a wrist-wearable device (e.g., wrist-wearable device 304; FIG. 3B), and/or a belt of the user 102. A compute core (e.g., the compute core 108a and/or 108b) may integrate a light emitter, an optical switch, a photo sensor, and control circuitry to manage and process data. A compute core may include control circuitry configured to manage its internal components including managing the light emitter which directs light through an optical switch to the fiber optic components. Fiber optic components may reflect a portion of the light back to a photo sensor within the compute core. A photo sensor within the compute core may be configured to receive the reflected light and provide data to control circuitry (e.g., a processor) in the compute core. The control circuitry may be configured to determine the pose of the user's hand and/or body part based on deltas between the original light emitted and the reflected light received from the fiber optic components.

An optical switch may be included in (e.g., integrated into) the compute core. The optical switch may include multiple inputs and outputs. The inputs of the optical switch may be optically coupled to the light emitter and the outputs may be optically coupled to the plurality of fiber optic components. The optical switch may be configured to direct the light through the various fiber optic components, allowing for selective activation and deactivation of specific pathways. The optical switch's configuration may be configured to improve (optimize) the device's responsiveness to user movements and environmental changes.

In some embodiments, a photo sensor is arranged in proximity with (e.g., adjacent to) the optical switch. The photo sensor may be arranged and configured to detect light that is reflected back through the fiber optic components. The photo sensor may be configured to capture variations in light intensity and patterns, which are indicative of the fiber optic components' curvature and the user's pose. In some embodiments, the photo sensor includes a Complementary Metal-Oxide-Semiconductor (CMOS) sensor, a spectrometer sensor, and/or a photonic circuit.

A compute core may include control circuitry interfacing with the photo sensor, optical switch, light emitter, and/or other components of the compute core. This circuitry may be programmed to analyze the data received from the photo sensor and determine (i) the curvature of the fiber optic components and, (ii) consequently, the user's pose. The control circuitry may be configured to process the information in real time, enabling the device to provide immediate feedback or adjustments based on the user's movements. In some embodiments, the data generated by the photo sensor is processed externally by a communicatively coupled device. For example, data from one or more photo sensors of the hand-wearable device 106 may be communicatively coupled to the compute core 108a such that control circuitry of the compute core 108a processes (e.g., analyzes and performs operations based on) the data from the one or more photo sensors.

In some embodiments, the wearable structure includes a power source configured to supply power to the light emitter, optical switch, photo sensor, and/or control circuitry. The power source may be configured to be compact and efficient, such that the device remains lightweight and unobtrusive while maintaining prolonged operational capability. In some embodiments, the power source includes a battery integrated into the compute core. The battery may be a circular shape to accommodate the circular nature of the compute core as shown in the wrist-wearable device (e.g., wrist-wearable device 304; FIG. 3B). In some embodiments, the power source and compute core are separate and arranged at different locations of the wearable structure. For example, the compute core may be on the portion of body-wearable device coupled to the user's back and the battery may be arranged on the user's belt.

The body-wearable device 104 and the hand-wearable device 106 each include a plurality of fiber optic components arranged along respective lengths of the user's body. For example, in FIG. 1A, fiber optic component 110a is arranged along the user's leg, fiber optic component 110b is arranged along the user's arm, and fiber optic component 110c is arranged along one of the user's phalanges. The fiber optic components are configured to receive light from a light emitter and reflect back portions of that light to be sensed by the photo sensor. Each fiber optic component 110 includes one or more passive sensors (e.g., passive sensors 114a and 114b), collectively referred to as passive sensors 114, along the length of the fiber optic component. The passive sensors 114 are configured to reflect a portion of light transmitted through the fiber optic component (e.g., each passive sensor may be configured to reflect a certain wavelength (or range of wavelengths) of light. The passive sensors 114 may include one or more fiber Bragg grating (FBG) sensing elements. The FBG sensing elements are microscopic wavelength-selective mirrors configured to reflect a specific wavelength (and allow transmission of) the rest of the optical signal that the light emitter generated. As shown in FIG. 1A, the passive sensors 114 are distributed along the path of each fiber optic component 110. In some embodiments, the passive sensors 114 are arranged at specific locations along the user's body. In some embodiments, the passive sensors 114 are arranged at regular intervals along the fiber optic components. The passive sensors are discussed further in reference to FIGS. 3A and 3B.

FIG. 1B illustrates the user 102 wearing the body-wearable device 104 and performing a movement at a second point in time. As the user 102 moves their body, one or more parameters of the fiber optic components 110 are adjusted (e.g., the fiber optic components are stretched, compressed, heated, etc.). For example, when the user 102 lifts their leg, this stretches the fiber optic component 110a which directly impacts the properties of the passive sensor 114a. Strain and/or temperature changes cause the passive sensor 114a to reflect back an adjusted wavelength of light to the photo sensor. The delta in the wavelength may be used to determine the pose/movement of the user 102. For example, when the user 102 raises their leg, a strain may be applied to the fiber optic component 110a, and light reflected back to the photo sensor by multiple sensors along the fiber optic component 110a may be used to generate a mesh of the user's leg. As an example, based on the quantity and/or wavelength of the reflected light, the control circuitry is able to accurately determine the position/orientation of the leg of the user 102. In FIG. 1B, the screen 112 illustrates the user's movements in real-time based on the sensed data.

The detection of the user's movements based on the reflected light in the plurality of fiber optic components 110 may occur continuously (e.g., at regular intervals) in real-time such that the system is able to determine/track the user's movement/pose. Fiber optic components can be very sensitive and accurate, but the data may require significant processing. The compute cores may be configured to handle the computation, and/or as mentioned above, the computation may be sent to another processing device to determine the pose of each limb based on raw data from the fiber optic components and/or pre-processed data from the compute core(s).

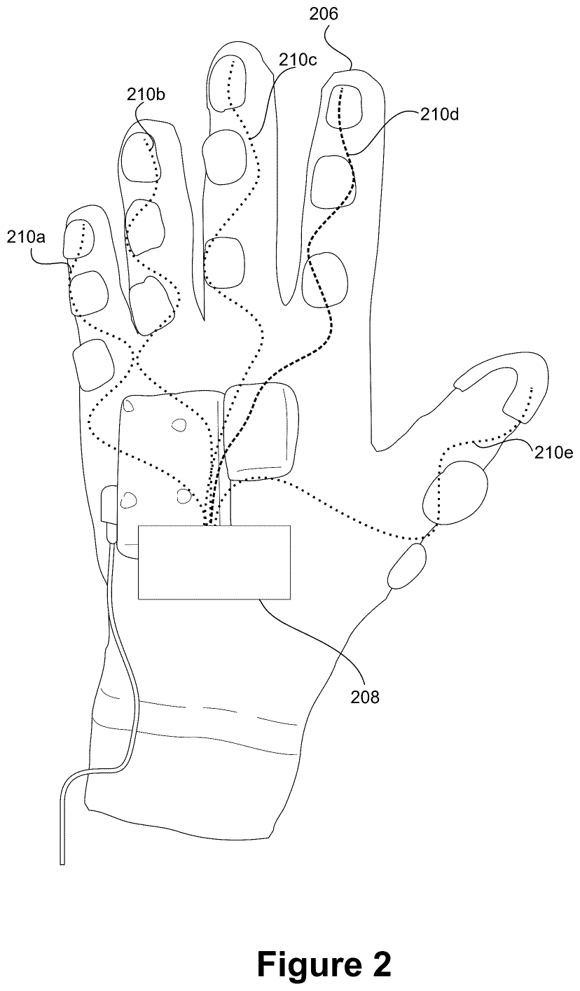

FIG. 2 illustrates an example wearable device (e.g., the hand-wearable device 106), that includes one or more fiber optic components, in accordance with some embodiments. FIG. 2 shows a glove 206 configured to be worn on the hand of a user (e.g., the user 102; FIGS. 1A and 1B), incorporating multiple fiber optic components (e.g., fiber optic components 210a-210e, collectively referred to as the plurality fiber optic components 210). in some embodiments, the fiber optic components 210 are instances of the fiber optic components 110. Each fiber optic component of the plurality of fiber optic components 210 is coupled to a portion of the glove 206 corresponding to the user's phalanges, e.g., to provide comprehensive coverage and precise motion capture. The sensor length of each respective fiber optic component that extends from the compute core 208 toward the fingertip of each respective finger may be a predetermined length (e.g., measuring between 25 and 30 centimeters). This configuration captures the range of motion from the user's fingertip to the wrist, e.g., encompassing a minimum of four joints. In some embodiments, the compute core 208 is an instance of a compute core 108 (e.g., the compute core 108a or 108b).

In some embodiments, each fiber optic component includes a plurality of passive sensors (e.g., FBG sensing elements). A higher passive sensor count can enhance precision (e.g., the resolution). In some embodiments, each fiber optic component includes 8 to 12 FBG sensing elements. In some embodiments, the FBG sensing elements are selected to reduce/minimize interpolation errors that affect wavelength sensitivity, e.g., with sizes ranging from 8 to 20 millimeters.

FIG. 2 further illustrates the compute core 208 coupled to a first end of each fiber optic component. In some embodiments, the compute core 208 includes an FBG interrogator using a Photonic Integrated Circuit (PIC). In some embodiments, the PIC integrates optical components such as waveguides, lasers, and detectors into a single substrate, thereby allowing for high-speed and low-power sensing. In some embodiments, the compute core is less than 5 inches in diameter, allowing it to be mounted on various wearable devices, such as the back of the glove 206, within a wrist-wearable device (e.g., wrist-wearable device 304; FIG. 3B), or a body-worn device (e.g., body-wearable device 104; FIG. 1A). In some embodiments, the compute core 208 (e.g., in conjunction with the wearable device) is configured to operate with low power, e.g., less than 5 W. The compute core 208 may be battery-powered for 2 to 6 hours, making it suitable for extended use in various applications.

In some embodiments, each fiber optic component 210 (e.g., fiber optic components 210a-210e) are equipped with at least 5 to 10 FBG sensing elements. For example, the fiber optic components may be routed through the fingers, allowing for detailed tracking of finger movements. In some embodiments, the fiber optic components 210 are arranged in a meandering pattern along each phalange as illustrated in FIG. 2. In some embodiments, the fiber optic components are multi-core components. In some embodiments, the fiber optic components are single-core components.

In some embodiments, the wearable structure and the compute core 208 of the wearable device (e.g., the glove 206) comprise a system that operates with an update rate between 60 to 200 Hz, e.g., ensuring frequent data refresh for accurate motion capture. In some embodiments, data latency is maintained at less than 5 milliseconds, with shape estimation latency under 10 milliseconds. These metrics are important for real-time operation and may be required for applications requiring immediate feedback, such as virtual reality or robotic teleoperation. Maintaining a balance between low-data latency and high-data refresh rates maintains the accuracy required for an uninterrupted user experience.

In some embodiments, each fiber optic component 210a-210e has a diameter of less than 1 millimeter. In some embodiments, each fiber optic component 210a-210e has a minimum bend radius of 5 millimeters or less. For example, fiber optic components that meet these specifications reduce/minimize encumbrance and enhance durability, allowing integration into the glove without adding significant bulk or weight. Additionally, the small diameter and flexible bend radius ensure user comfort and the glove's longevity.

The fiber optic components may be configured to withstand the demands of haptic feedback and data capture applications. For example, coatings may be applied to the fiber optic components 210a-210e to reduce/minimize stiffness, thereby reducing power requirements. In some embodiments, the wearable device is configured to operate for at least two hours at a 60 Hz update rate, ensuring extended use without frequent recharging.

FIGS. 3A and 3B illustrate example wearable devices including one or more fiber optic components, in accordance with some embodiments. FIG. 3A illustrates a compute core 308 (e.g., an instance of any of the compute cores described herein), one or more fiber optic components 310a-310e (e.g., an instance of any of the fiber optic components described herein), and passive sensors 312 (e.g., FBG sensing elements) including passive sensors 312a-312g. The passive sensors 312 are distributed along each respective fiber optic component 310 (e.g., at particular locations corresponding to a user anatomy or at regular locations).

FIG. 3B illustrates a wrist-wearable device 304 (e.g., a smartwatch) worn by a user 302 (e.g., the user 102). The wrist-wearable device 304 may be an instance of one of the wearable devices described herein. Fiber optic components 310a-310e are coupled to, or components of, the wrist-wearable device 304. In some embodiments, each fiber optic component 310 is coupled to a respective phalange, limb, or digit. For example, fiber optic component 310e is coupled to the user's thumb. In some embodiments, one end of each fiber optic component 310 is coupled to the tip of each respective digit (e.g., finger or thumb), e.g., without coupling to a wearable structure. In some embodiments, a PIC within the compute core 308 operates by probing each finger's passive sensors with a tunable laser and measuring the reflected light using a photodetector (e.g., the photo sensor discussed previously with respect to FIGS. 1A-1B). This data may be converted into bend angles, allowing for the reconstruction of a 3D shape corresponding to a portion of the user's body.

In some embodiments, a pose estimation algorithm is used to determine a pose for at least a portion of a user's body (e.g., legs, full body, fingers, wrist, limbs, etc.). In some embodiments, determining the pose includes estimating bone lengths (e.g., for fingers/limbs) without additional (external) sensor data. Control circuitry (e.g., one or more processors) in a compute core (e.g., compute core 108, 208, etc.) may execute the pose estimation algorithm. In some embodiments, each fiber optic component's shape (e.g., fiber optic components 210) is registered to a common origin, allowing an objective function to reduce/minimize errors between the fiber optic component shape and the corresponding tunnel embedded in the wearable structure (e.g., the fiber optic component 210 arranged in a meandering pattern in FIG. 2). In some embodiments, the pose estimation algorithm comprises an inverse kinematic algorithm. In some embodiments, once an origin is determined, the pose estimation algorithm determines the pose of a hand mesh, e.g., using curve shape constraints to best fit the mesh to the input data. This approach allows for accurate pose estimation even under conditions of occlusion and external interference. In some embodiments, the origin corresponds to where the compute core is located, such as the back of the user's hand coupled to the glove, on the user's back, or on the user's belt.

In some embodiments, the pose estimation algorithm applies inverse kinematics calculations. Inverse kinematics calculates the joint movements needed for an articulated structure to reach a specific position and orientation in space. Unlike forward kinematics, which computes the end position from known joint angles, inverse kinematics works in reverse by solving complex equations to find joint configurations that achieve a desired goal. This process enables precise motion, which can be computationally challenging. At a high level, the pose estimation algorithm uses the data collected by the compute core of the wearable structure. The pose estimation algorithm determines a wearable structure mesh that relates to the position of the user's body parts coupled to the wearable structure at a specific point in time (e.g., the user's body as shown in FIG. 1A). When the user moves part of their body, the reflected light from the passive sensors to the compute core changes. In some embodiments, the compute core determines a delta between the light transmitted and the light reflected back, and, using the pose estimation algorithm and inverse kinematics, the precise movement made by the user (e.g., at a joint level) is determined and stored and/or displayed to the user, as shown in FIGS. 1A and 1B. In some embodiments, the pose estimation algorithm is stored on a server communicatively coupled with the compute core 108 and/or another communicatively coupled device such as a smart phone, AR headset, etc.

The wearable devices and pose-estimation applications extend beyond gloves to other body tracking systems such as full-body tracking systems, e.g., in which the optic fibers are placed on each limb, with an origin fixture located on the body, such as on the user's back. This capability allows for comprehensive body motion capture without the need for external cameras, making it ideal for use in environments where traditional motion capture systems are impractical.

The accuracy provided by the fiber optics in the wearable devices is particularly useful for gathering training data for teaching robots complex tasks. The wearable device (e.g., glove, bodysuit, etc.) can be used for teleoperation and/or data collection, thereby providing a rich dataset for training AI models to mimic human movements. The system's ability to track movements with high precision and minimal interference makes it a valuable tool for developing advanced robotic systems and enhancing human-robot interaction.

For example, when training a home robot to carefully handle a wine glass in a dishwasher, the glove captures precise hand movements and grip strength, ensuring the robot can replicate the delicate task without breaking the glass. This data-driven approach allows the robot to learn the nuances of human touch and dexterity. Similarly, when using the body suit, a user can demonstrate the exact leg lift and body movements required to navigate stairs. The suit captures detailed motion data, including the angle and height of each step, enabling robots to learn and mimic these actions accurately. This level of precision in data collection is crucial for developing robots capable of performing everyday tasks with human-like efficiency and care, ultimately enhancing their ability to assist in domestic environments.

In some embodiments, the wearable device is designed and configured to capture and record detailed human behavior, e.g., providing invaluable data for analysis and modeling. By utilizing advanced fiber optic components, the device accurately tracks movements and gestures, allowing for a comprehensive understanding of human actions. This data can be used to study behavioral patterns, improve ergonomic designs, and develop more intuitive human-machine interfaces. The precise motion capture capabilities ensure that even subtle movements are recorded, offering a rich dataset for researchers and developers aiming to enhance human-computer interaction.

In addition to recording behavior, the wearable devices described herein may be used for teleoperation, allowing users to control devices remotely with precision and accuracy. For example, a wearable device may be configured to translate the user's movements into real-time commands. This wearable technology facilitates seamless interaction with remote systems, such as robotic arms or drones. This capability is particularly beneficial in environments where direct human presence is impractical or hazardous. The device's high sensitivity and low latency allow for remote operations to be smooth and responsive, providing users with a sense of direct control over distant machinery. This opens up new possibilities for remote work, exploration, and assistance in various fields.

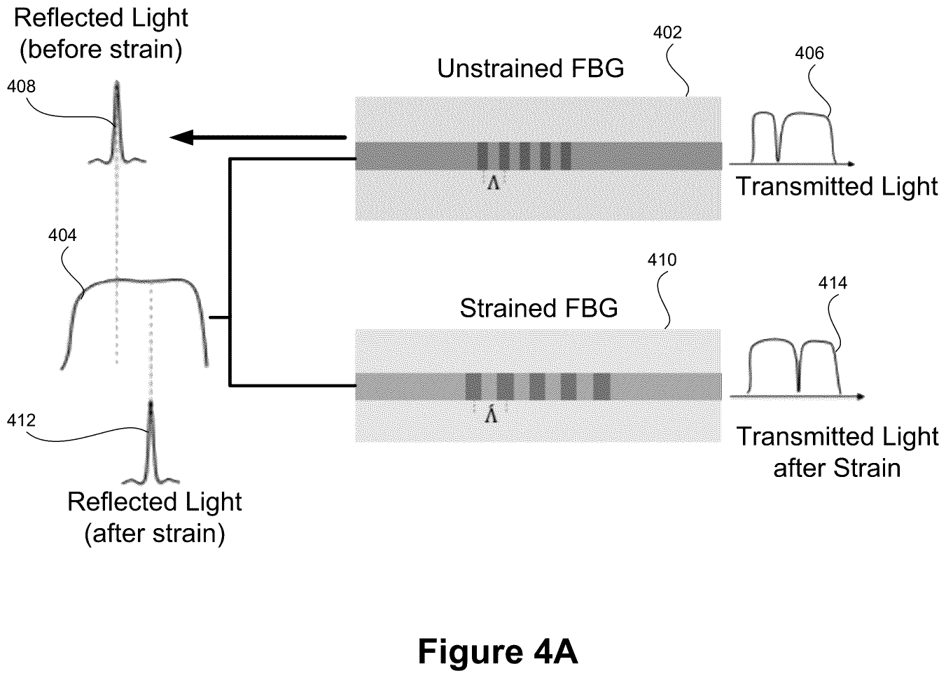

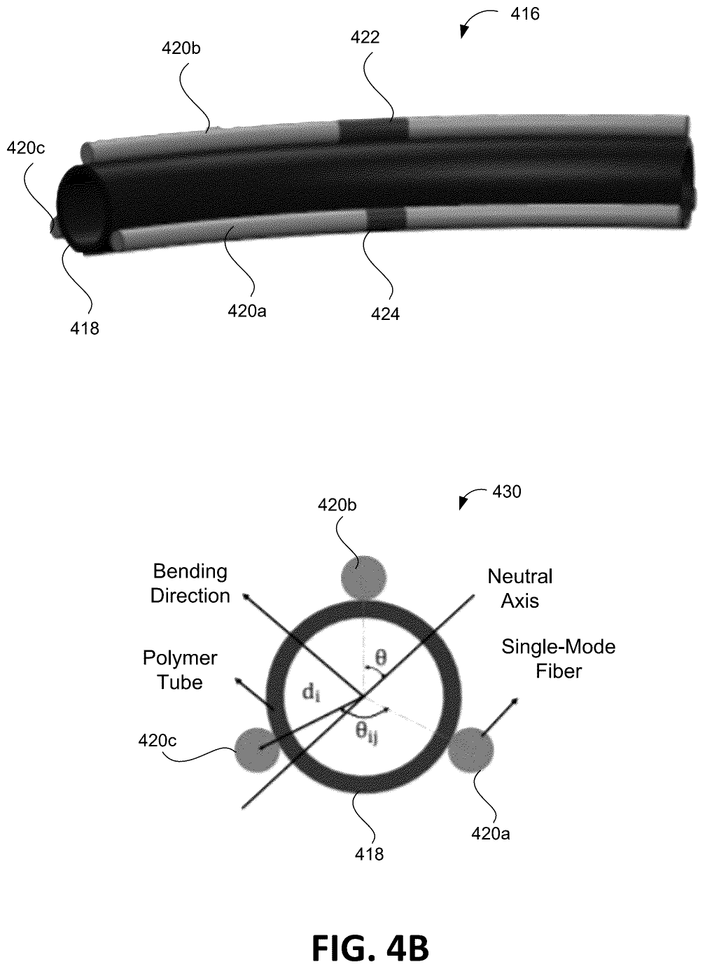

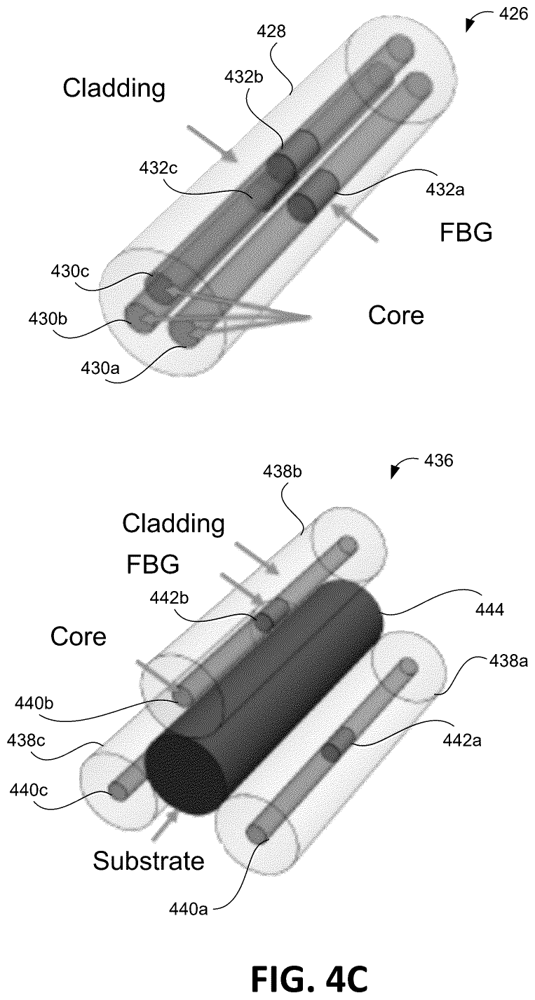

FIGS. 4A-4C illustrate fiber Bragg grating principles and example configurations of the fiber optic components, in accordance with some embodiments. In some embodiments, an FBG sensing element is a periodic variation of the core refractive index. An FBG sensing element shows large reflectivity around a certain wavelength which fulfills the Bragg condition. External perturbation (temperature/mechanical strain) can change the grating period, which causes a shift in the reflected signal as shown in FIG. 4A. The mechanical strain may be caused by a user moving, flexing, and/or bending portions of the user's body.

FIG. 4A illustrates a working principle of an FBG sensing element and how it responds to external strain by shifting the reflected light wavelength. For example, FIG. 4A shows a segment of optical fiber (e.g., corresponding to an unstrained FBG sensing element 402), containing a regular periodic grating that is depicted by the alternating light and dark bands. The unstrained FBG sensing element 402 contains a grating period of A. When incident light 404 that contains a spectrum of wavelengths enters the fiber optic component, the unstrained FBG sensing element 402 reflects a narrow band of light (e.g., reflected light 408) centered in the Bragg wavelength, while allowing the rest of the light to transmit (e.g., transmitted light 406). As shown in FIG. 4A, the reflected light 408 signal appears as a sharp peak in the spectral plot labeled reflected light (before strain) and the transmitted signal shows a corresponding dip at that same wavelength.

FIG. 4A further illustrates a strained FBG sensing element 410. For example, strain on the FBG sensing element may be caused by a user flexing their finger, moving a part of their body, or any action that bends or stretches the passive sensors (e.g., the FBG sensors). The same FBG sensing element in the unstrained FBG sensing element 402 is not subjected to the mechanical strain applied to the strained FBG sensing element 410. The mechanical strain causes the grating period to increase to A′. The change in period shifts the Bragg wavelength to a longer wavelength, shown by a rightward shift of the reflected light peak (e.g., reflected light 412). The reflected signal changes as indicated by the reflected light 412, and the transmitted light 414 also shifts accordingly as seen in the transmitted light after strain plot.

FBG sensing elements are sensitive to strain and temperature, which makes them useful for high-accuracy sensing applications. Changes in the grating period shift the reflected wavelength. The spectral shift in the reflected light is a measurable output used to detect physical changes. The diagram shown in FIG. 4A illustrates how external perturbations modulate the fiber optic components' optical properties, allowing FBG sensing elements to act as precise passive sensors. For example, when user 102 raises their leg in FIG. 1B, some of the FBG sensors are stretched, increasing the grating period, and some of the FBG sensors are compressed, decreasing the grating period. The deltas in the grating periods change the amount of light reflected back to the photo sensor. Thus, changes in the reflected light from each respective passive sensor (e.g., FBG sensor) provide the raw data used by a compute core to determine changes in the user's position.

In an example where the incident light 404 is white light, white light contains the entire color spectrum including many different wavelengths. If white light is sent down the unstrained FBG sensing element 402, the reflected light 408 signal may include a single color reflected, while every other color in the spectrum is transmitted through as the transmitted light 406.

FIG. 4B illustrates a 3D-shape sensing probe 416 comprising a polymer tube 418 and multiple single-mode optical fiber components (e.g., optical fiber components 420a-420c), collectively referred to as the optical fiber components 420. Each respective single-mode optical fiber component includes at least one passive sensor (e.g., FBG sensing element). In the 3D-shape sensing probe 416, the FBG sensing elements (e.g., a FBG sensing element 422 and a FBG sensing element 424) are placed symmetrically around the circumference at each axial (z) position. When the 3D-shape sensing probe 416 bends, the optical fiber component 420a on the inner side of the curve experiences compression which shortens the wavelength (e.g., as described above). The two outer side optical fiber components 420b and 420c experience tension and lengthened wavelength. The differential strain response between each respective optical fiber component of the optical fiber components 420 enables the compute core to determine the bending direction and curvature by comparing the wavelength shift of all three FBG sensing elements, including first and second FBG sensing elements 422 and 424.

FIG. 4B further illustrates a cross-sectional view 430 of the 3D-shape sensing probe 416. The neutral axis experiences negligible strain during bending, while the single-mode optical fiber components 420 (labeled by positions and angle θᵢⱼ) are embedded at different distances dᵢ from this axis. The strain Eᵢ in each fiber may include:

$${E}_{t} , the strain due to temperature \qquad ( 1 )$$

$${kd}_{i} sin ( θ + {θ}_{ij} ) , the component due to bending \qquad ( 2 )$$

These equations enable the calculation of curvature (k), the bending angle (theta), and the temperature effects. By solving the strain values using the FBG-measured wavelength shifts (Δλᵢ), the 3D shape of the fiber (e.g., the shape of the user's hand) can be reconstructed, e.g., using the Frenet-Serret equations. The distance dᵢ (inter-core spacing) affects sensitivity to bending, making geometry choice an important component for accurate 3D-shape sensing.

FIG. 4C illustrates a multi-core fiber (MCF) structure 426 usable in fiber optic sensing applications. The MCF structure 426 includes cores 430a-430c within a common cladding layer 428, with FBG sensing elements 432a-432c arranged in the cores for sensing purposes. This structure allows for multiple cores (e.g., 4 or 7) arranged symmetrically within the MCF structure 426. In some embodiments, the MCF structure 426 has a predetermined diameter (e.g., 125 μm diameter). By using multiple cores, the sensor benefits from redundant measurements, which can improve accuracy and allow noise averaging, enhancing the robustness of 3D shape or strain sensing.

FIG. 4C further illustrates an alternative fiber optic sensing configuration 436. In this approach, multiple single-core fibers 440a-440c are arranged around a common substrate 444, e.g., to maintain a fixed geometry. In some embodiments, each single-core fiber 440a-440c includes its own core, cladding 438a-438c, and inscribed sensors 442a-442b (e.g., FBG sensing elements) for strain and/or temperature sensing.

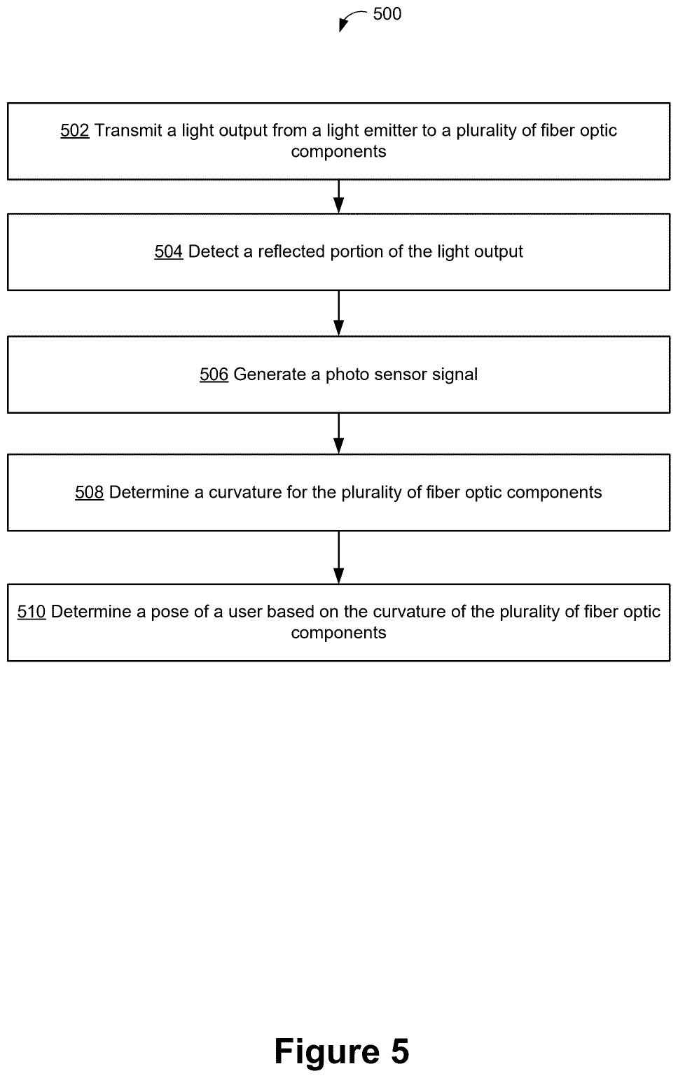

FIG. 5 illustrates a flow diagram for a method 500 of pose estimation, in accordance with some embodiments. Operations (e.g., steps) of the method 500 may be performed by one or more processors (e.g., central processing unit and/or MCU) of a wearable device. At least some of the operations shown in FIG. 5 correspond to instructions stored in a computer memory or computer-readable storage medium (e.g., storage, RAM, and/or memory) of the wearable device. Operations of the method 500 can be performed by a single device alone or in conjunction with one or more processors and/or hardware components of another communicatively coupled device (e.g., a glove, a wrist-wearable device, a body suit, etc.) and/or instructions stored in memory or computer-readable medium of the other device communicatively coupled to the system. In some embodiments, the various operations of the methods described herein are interchangeable and/or optional, and respective operations of the methods are performed by any of the aforementioned devices, systems, or combination of devices and/or systems. For convenience, the method operations will be described below as being performed by particular component or device but should not be construed as limiting the performance of the operation to the particular device in all embodiments.

A device with a wearable structure that includes a light emitter, fiber optic components, an optical switch, and a photo sensor is disclosed herein. The device uses control circuitry to analyze light reflections from one or more fiber optic components to determine the user's pose, powered by an integrated power source.

(A1) The method 500 occurs at a wearable device (e.g., any of the wearable devices described herein) with one or more components including a light emitter, an optical switch, a photo sensor, and one or more fiber optic components. The method 500 includes, transmitting (502) a light output from a light emitter to a plurality of fiber optic components. In some embodiments, the light output comprises white light. In some embodiments, the light output is configured to include a range of wavelengths. In some embodiments, the light emitter is coupled to the fiber optic components via an optical switch (e.g., the light emitter is selectively coupled to different subsets of the fiber optic components according to the state of the optical switch).

The method 500 includes detecting (504), via a photo sensor, a reflected portion of the light output, where the reflected portion is reflected by one or more passive sensors (e.g., FBG sensing elements) within the plurality of fiber optic components. In some embodiments, each passive sensor is configured to reflect a different portion of the range of wavelengths included in the light output. In some embodiments, the photo sensor is coupled to the fiber optic components via an optical switch (e.g., the photo sensor is selectively coupled to different subsets of the fiber optic components according to the state of the optical switch).

The method 500 includes generating (506), via the photo sensor, a photo sensor signal based on the reflected portion of the light output. In some embodiments, the photo sensor generates an electrical signal that indicates characteristics of the reflected portion of the light output (e.g., indicates a wavelength, intensity, peak, median, mean, mode, and/or tail of the reflected portion).

The method 500 includes determining (508) a curvature for the plurality of fiber optic components based on the photo sensor signal. For example, the curvature of the fiber optic components is determined based on delta between reflected portions of light from multiple fiber optic components and/or sensors. In some embodiments, the curvature of the fiber optic components is determined based on a comparison between expected reflections and actual reflections from sensors of the fiber optic components.

The method 500 includes determining (510) a pose of a user based on the curvature of the plurality of fiber optic components. In some embodiments, the pose of the user is determined using a pose estimation algorithm (e.g., an inverse kinematics algorithm).

(A2) In some embodiments of A1, the curvature for the plurality of fiber optic components is determined by a processor (and/or other type of control circuitry) of the wearable device that comprises the light emitter, the photo sensor, and the plurality of fiber optic components.

(A3) In some embodiments of any of A1-A2, the pose of the user is determined by a processor (and/or other type of control circuitry) of the wearable device that comprises the light emitter, the photo sensor, and the plurality of fiber optic components.

(A4) In some embodiments of any of A1-A3, the curvature for the plurality of fiber optic components is determined based on a plurality of photo sensor signals, each photo sensor signal of the plurality of photo sensor signals corresponding to a different passive sensor within the plurality of fiber optic components.

(A5) In some embodiments of any of A1-A4, the pose of the user is determined by mapping the curvature to human skeletal data. For example, the human skeletal data may comprise human anatomy data and human mobility data. In some embodiments, a kinematics algorithm is used to determine the pose of the user. For example, the human skeletal data may comprise human anatomy data and human mobility data. In some embodiments, a kinematics algorithm is used to determine the pose of the user.

(B1) In accordance with some embodiments, a device includes a plurality of fiber optic components and a wearable structure (e.g., a glove 206) configured to couple to a body of a user (e.g., user 102). The wearable structure includes a light emitter (e.g., an LED) coupled to the plurality of fiber optic components (e.g., fiber optic components 210a-210e) and is configured to transmit a light output to the plurality of fiber optic components. The wearable structure further includes an optical switch that is coupled to the plurality of fiber optic components and has a plurality of inputs and one or more outputs. The wearable structure further includes a photo sensor (e.g., a CMOS sensor) that is coupled to at least one output of the optical switch, where the photo sensor is configured to detect a reflected portion of the light output from the light emitter and output a corresponding photo sensor signal. The wearable structure further includes control circuitry (e.g., a PIC) that is coupled to the photo sensor and configured to determine (e.g., via a pose estimation algorithm) a curvature of a respective fiber optic component of the plurality of fiber optic components based on analysis of the corresponding photo sensor signal, and determines a pose of the user based on the curvature of the respective fiber optic component. The wearable structure further includes a power source (e.g., a battery) configured to provide power to the control circuitry, the light emitter, the optical switch, and the photo sensor. Each fiber optic component of the plurality of fiber optic components includes a respective set of passive sensors, each passive sensor of the respective set of passive sensors is configured to reflect a portion of light transmitted to the fiber optic component.

In some embodiments, the wearable structure comprises a compute component (e.g., a compute core 108) and one or more of the light emitter, the optical switch, the photo sensor, the control circuitry, and the power source are components of the compute component. In some embodiments, the light emitter comprises an LED. The control circuitry may comprise one or more processors, a processing unit, a microprocessor, an FPGA, and/or other types of control circuitry. In some embodiments, the photo sensor comprises a CMOS sensor, a spectrometer, and/or a photonic circuit. For example, the photo sensor may receive the light reflected by the passive (optical) sensors and detect changes in a user's hand pose by detecting changes in the colors reflected back by the sensors. This allows the device to measure the bend and curvature precisely. In some embodiments, the optical switch comprises a connector for each fiber optic component of the plurality of fiber optic components. For example, the optical switch may include 5 connectors for 5 fiber optic components (e.g., where each fiber optic component is positioned along a different finger of a user's hand. In some embodiments, the wearable structure comprises a puck. In some embodiments, the puck diameter is less than 5 inches (e.g., 3 inches). In some embodiments, the power source is a battery. In some embodiments, the power source is separate from the wearable structure. In some embodiments, the power source and the wearable structure are coupled to different portions of the user's body. For example, the wearable structure may be coupled to a user's hand (e.g., is a component of a glove) and the power source may be coupled to the user's head (e.g., is a component of a headset). The features are further discussed in FIGS. 1A-5.

FIG. 2 illustrates a glove 206 configured to be worn on the hand of a user that includes multiple fiber optic components 210a-210e collectively referred to as the fiber optic components 210. In some embodiments, at least one fiber optic component 210 is coupled to the portion of the glove 206 covering each of the user's phalanges. For example, the fiber optic component 210a is coupled to the portion of the glove 206 coupled to the user's pinky finger, the fiber optic component 210b is coupled to the portion of the glove 206 coupled to the user's ring finger, the fiber optic component 210c is coupled to the portion of the glove 206 coupled to the user's middle finger, the fiber optic component 210d is coupled to the portion of the glove 206 coupled to the user's pointer finger, and the fiber optic component 210e is coupled to the portion of the glove 206 coupled to the user's thumb.

(B2) In some embodiments of B1, the respective set of passive sensors comprises a set of FBG sensors. In some embodiments, the set of FBG sensors is configured to form a sensing element. Each FBG sensor may comprise a microscopic, wavelength-selective mirror adapted to reflect a specific wavelength (or band of wavelengths) and transmit the rest of the optical signal. In some embodiments, the FBG sensors are arranged at regular intervals. In some embodiments, the set of FBG sensors is arranged at positions corresponding to respective joints of the user's body. In some embodiments, the set of passive sensors comprises a set of non-line-of-sight sensors. In some embodiments, the number of passive sensors per length on the fiber optic components determines the resolution and accuracy of the output of the data detected by the photo sensor.

(B3) In some embodiments of any of B1-B2, each passive sensor of the respective set of passive sensors is configured to reflect a respective predetermined wavelength. As described in FIGS. 1A-4C, the passive sensors (e.g., FBG sensing elements 312a-312g) are FBG sensors.

(B4) In some embodiments of any of B1-B3, at least one set of passive sensors is arranged in a meandering pattern extending from the wearable structure toward an end of a respective limb of the user. For example, a meandering pattern along the length of the phalange of the user's finger is configured to accommodate a stretch/extension of the user's hand as illustrated and described in FIG. 2.

(B5) In some embodiments of any of B1-B4, at least one set of passive sensors is arranged in a linear pattern extending from the wearable structure toward an end of a respective limb of the user.

(B6) In some embodiments of any of B1-B5, each fiber optic component of the plurality of fiber optic components is arranged to extend from the wearable structure along a different part of the body of the user. For example, a fiber optic component may extend along a length of a limb of the user (e.g., along the user's arm or leg). In another example, each fiber optic component may extend along a different finger of the user. As a specific example, the wearable structure may be arranged on a wrist of the user and each fiber optic component may extend from the wearable structure along a different finger of the user. The wearable structure may be arranged at other locations along the user's body (e.g., the user's back, chest, waist, or neck).

(B7) In some embodiments of any of B1-B6, the device comprises a wearable glove, a wrist-wearable device, or a belt. In some embodiments, the device comprises a smartwatch. In some embodiments, the device comprises an article of clothing. In some embodiments, the device comprises a bodysuit.

(B8) In some embodiments of any of B1-B7, the plurality of fiber optic components are embedded in a material of the device. In some embodiments, the plurality of fiber optic components extend from the wearable structure. In some embodiments, the plurality of fiber optic components comprise a mesh representative of the wearable structure.

(B9) In some embodiments of any of B1-B8, the control circuitry is configured to receive one or more additional photo sensor signals from one or more photo sensors that are not components of the wearable structure, wherein the pose of the user is further based on the one or more additional photo sensor signals. In some embodiments, the control circuitry is not a component of the wearable structure. For example, the photo sensor signal may be transmitted from the wearable structure to control circuitry that is remote from the wearable structure. In some embodiments, the control circuitry is configured to determine the curvature information and transmit the curvature information to a remote system (e.g., device) to determine the pose of the user. For example, the control circuitry may perform some pre-processing and a different processor (remote from the device) performs the pose determinations based on the pre-processed data.

(B10) In some embodiments of any of B1-B9, the light emitter, the optical switch, and the photo sensor are components of a photonic integrated circuit.

(B11) In some embodiments of any of B1-B10, the light emitter comprises a laser component. For example, a tunable laser is used to probe each fiber optic component, and a photodetector is used to measure the corresponding reflected light.

(C1) In accordance with some embodiments, a non-transitory computer readable storage medium including instructions that, when executed by control circuitry of a wearable device, cause the wearable device to perform one or more operations. The one or more operations include transmitting a light output from a light emitter to a plurality of fiber optic components and detecting, via a photo sensor, a reflected portion of the light output, where the reflected portion is reflected by one or more passive sensors within the plurality of fiber optic components. The one or more operations include generating, via the photo sensor, a photo sensor signal based on the reflected portion of the light output, determining a curvature for the plurality of fiber optic components based on the photo sensor signal, and determining a pose of a user based on the curvature of the plurality of fiber optic components.

(C2) In some embodiments of C1, the pose of the user is determined by mapping the curvature to human skeletal data.

(C3) In some embodiments of any of C1-C2, the pose of the user is determined using a kinematics algorithm.

(C4) In some embodiments of any of C1-C3, the light emitter and the photo sensor are components of a photonic integrated circuit.

In accordance with some embodiments, a system that includes a wrist-wearable device (or a plurality of wrist-wearable devices) and a pair of AR glasses, and the system is configured to perform operations corresponding to any of A1-A5.

In accordance with some embodiments, a non-transitory computer readable storage medium including instructions that, when executed by a computing device in communication with a pair of augmented-reality glasses, cause the computer device to perform operations corresponding to any of A1-A5.

In accordance with some embodiments, a method of operating a pair of augmented-reality glasses, including operations that correspond to any of A1-A5.

The devices described above are further detailed below, including wrist-wearable devices, headset devices, systems, and haptic feedback devices. Specific operations described above may occur as a result of specific hardware, such hardware is described in further detail below. The devices described below are not limiting and features on these devices can be removed or additional features can be added to these devices.

### Example Extended-Reality Systems

FIGS. 6A 6B, 6C-1, and 6C-2, illustrate example XR systems that include AR and MR systems, in accordance with some embodiments. FIG. 6A shows a first AR system 600a and first example user interactions using a wrist-wearable device 626, a head-wearable device (e.g., AR device 628), and/or a HIPD 642. FIG. 6B shows a second AR system 600b and second example user interactions using a wrist-wearable device 626, AR device 628, and/or an HIPD 642. FIGS. 6C-1 and 6C-2 show a third MR system 600c and third example user interactions using a wrist-wearable device 626, a head-wearable device (e.g., an MR device such as a VR device), and/or an HIPD 642. As the skilled artisan will appreciate upon reading the descriptions provided herein, the above-example AR and MR systems (described in detail below) can perform various functions and/or operations.

The wrist-wearable device 626, the head-wearable devices, and/or the HIPD 642 can communicatively couple via a network 625 (e.g., cellular, near field, Wi-Fi, personal area network, wireless LAN). Additionally, the wrist-wearable device 626, the head-wearable device, and/or the HIPD 642 can also communicatively couple with one or more servers 630, computers 640 (e.g., laptops, computers), mobile devices 650 (e.g., smartphones, tablets), and/or other electronic devices via the network 625 (e.g., cellular, near field, Wi-Fi, personal area network, wireless LAN). Similarly, a smart textile-based garment, when used, can also communicatively couple with the wrist-wearable device 626, the head-wearable device(s), the HIPD 642, the one or more servers 630, the computers 640, the mobile devices 650, and/or other electronic devices via the network 625 to provide inputs.

Turning to FIG. 6A, a user 602 is shown wearing the wrist-wearable device 626 and the AR device 628 and having the HIPD 642 on their desk. The wrist-wearable device 626, the AR device 628, and the HIPD 642 facilitate user interaction with an AR environment. In particular, as shown by the first AR system 600a, the wrist-wearable device 626, the AR device 628, and/or the HIPD 642 cause presentation of one or more avatars 604, digital representations of contacts 606, and virtual objects 608. As discussed below, the user 602 can interact with the one or more avatars 604, digital representations of the contacts 606, and virtual objects 608 via the wrist-wearable device 626, the AR device 628, and/or the HIPD 642. In addition, the user 602 is also able to directly view physical objects in the environment, such as a physical table 629, through transparent lens(es) and waveguide(s) of the AR device 628. Alternatively, an MR device could be used in place of the AR device 628 and a similar user experience can take place, but the user would not be directly viewing physical objects in the environment, such as table 629, and would instead be presented with a virtual reconstruction of the table 629 produced from one or more sensors of the MR device (e.g., an outward facing camera capable of recording the surrounding environment).

The user 602 can use any of the wrist-wearable device 626, the AR device 628 (e.g., through physical inputs at the AR device and/or built-in motion tracking of a user's extremities), a smart-textile garment, externally mounted extremity tracking device, the HIPD 642 to provide user inputs, etc. For example, the user 602 can perform one or more hand gestures that are detected by the wrist-wearable device 626 (e.g., using one or more EMG sensors and/or IMUs built into the wrist-wearable device) and/or AR device 628 (e.g., using one or more image sensors or cameras) to provide a user input. Alternatively, or additionally, the user 602 can provide a user input via one or more touch surfaces of the wrist-wearable device 626, the AR device 628, and/or the HIPD 642, and/or voice commands captured by a microphone of the wrist-wearable device 626, the AR device 628, and/or the HIPD 642. The wrist-wearable device 626, the AR device 628, and/or the HIPD 642 include an artificially intelligent digital assistant to help the user in providing a user input (e.g., completing a sequence of operations, suggesting different operations or commands, providing reminders, confirming a command). For example, the digital assistant can be invoked through an input occurring at the AR device 628 (e.g., via an input at a temple arm of the AR device 628). In some embodiments, the user 602 can provide a user input via one or more facial gestures and/or facial expressions. For example, cameras of the wrist-wearable device 626, the AR device 628, and/or the HIPD 642 can track the user 602's eyes for navigating a user interface.

The wrist-wearable device 626, the AR device 628, and/or the HIPD 642 can operate alone or in conjunction to allow the user 602 to interact with the AR environment. In some embodiments, the HIPD 642 is configured to operate as a central hub or control center for the wrist-wearable device 626, the AR device 628, and/or another communicatively coupled device. For example, the user 602 can provide an input to interact with the AR environment at any of the wrist-wearable device 626, the AR device 628, and/or the HIPD 642, and the HIPD 642 can identify one or more back-end and front-end tasks to cause the performance of the requested interaction and distribute instructions to cause the performance of the one or more back-end and front-end tasks at the wrist-wearable device 626, the AR device 628, and/or the HIPD 642. In some embodiments, a back-end task is a background-processing task that is not perceptible by the user (e.g., rendering content, decompression, compression, application-specific operations), and a front-end task is a user-facing task that is perceptible to the user (e.g., presenting information to the user, providing feedback to the user). The HIPD 642 can perform the back-end tasks and provide the wrist-wearable device 626 and/or the AR device 628 operational data corresponding to the performed back-end tasks such that the wrist-wearable device 626 and/or the AR device 628 can perform the front-end tasks. In this way, the HIPD 642, which has more computational resources and greater thermal headroom than the wrist-wearable device 626 and/or the AR device 628, performs computationally intensive tasks and reduces the computer resource utilization and/or power usage of the wrist-wearable device 626 and/or the AR device 628.

In the example shown by the first AR system 600a, the HIPD 642 identifies one or more back-end tasks and front-end tasks associated with a user request to initiate an AR video call with one or more other users (represented by the avatar 604 and the digital representation of the contact 606) and distributes instructions to cause the performance of the one or more back-end tasks and front-end tasks. In particular, the HIPD 642 performs back-end tasks for processing and/or rendering image data (and other data) associated with the AR video call and provides operational data associated with the performed back-end tasks to the AR device 628 such that the AR device 628 performs front-end tasks for presenting the AR video call (e.g., presenting the avatar 604 and the digital representation of the contact 606).

In some embodiments, the HIPD 642 can operate as a focal or anchor point for causing the presentation of information. This allows the user 602 to be generally aware of where information is presented. For example, as shown in the first AR system 600a, the avatar 604 and the digital representation of the contact 606 are presented above the HIPD 642. In particular, the HIPD 642 and the AR device 628 operate in conjunction to determine a location for presenting the avatar 604 and the digital representation of the contact 606. In some embodiments, information can be presented within a predetermined distance from the HIPD 642 (e.g., within five meters). For example, as shown in the first AR system 600a, virtual object 608 is presented on the desk some distance from the HIPD 642. Similar to the above example, the HIPD 642 and the AR device 628 can operate in conjunction to determine a location for presenting the virtual object 608. Alternatively, in some embodiments, presentation of information is not bound by the HIPD 642. More specifically, the avatar 604, the digital representation of the contact 606, and the virtual object 608 do not have to be presented within a predetermined distance of the HIPD 642. While an AR device 628 is described working with an HIPD, an MR headset can be interacted with in the same way as the AR device 628.

User inputs provided at the wrist-wearable device 626, the AR device 628, and/or the HIPD 642 are coordinated such that the user can use any device to initiate, continue, and/or complete an operation. For example, the user 602 can provide a user input to the AR device 628 to cause the AR device 628 to present the virtual object 608 and, while the virtual object 608 is presented by the AR device 628, the user 602 can provide one or more hand gestures via the wrist-wearable device 626 to interact and/or manipulate the virtual object 608. While an AR device 628 is described working with a wrist-wearable device 626, an MR headset can be interacted with in the same way as the AR device 628.

### Integration of Artificial Intelligence with XR Systems

FIG. 6A illustrates an interaction in which an artificially intelligent virtual assistant can assist in requests made by a user 602. The AI virtual assistant can be used to complete open-ended requests made through natural language inputs by a user 602. For example, in FIG. 6A the user 602 makes an audible request 644 to summarize the conversation and then share the summarized conversation with others in the meeting. In addition, the AI virtual assistant is configured to use sensors of the XR system (e.g., cameras of an XR headset, microphones, and various other sensors of any of the devices in the system) to provide contextual prompts to the user for initiating tasks.

FIG. 6A also illustrates an example neural network 652 used in Artificial Intelligence applications. Uses of Artificial Intelligence (AI) are varied and encompass many different aspects of the devices and systems described herein. AI capabilities cover a diverse range of applications and deepen interactions between the user 602 and user devices (e.g., the AR device 628, an MR device 632, the HIPD 642, the wrist-wearable device 626). The AI discussed herein can be derived using many different training techniques. While the primary AI model example discussed herein is a neural network, other AI models can be used. Non-limiting examples of AI models include artificial neural networks (ANNs), deep neural networks (DNNs), convolution neural networks (CNNs), recurrent neural networks (RNNs), large language models (LLMs), long short-term memory networks, transformer models, decision trees, random forests, support vector machines, k-nearest neighbors, genetic algorithms, Markov models, Bayesian networks, fuzzy logic systems, and deep reinforcement learnings, etc. The AI models can be implemented at one or more of the user devices, and/or any other devices described herein. For devices and systems herein that employ multiple AI models, different models can be used depending on the task. For example, for a natural-language artificially intelligent virtual assistant, an LLM can be used and for the object detection of a physical environment, a DNN can be used instead.

In another example, an AI virtual assistant can include many different AI models and based on the user's request, multiple AI models may be employed (concurrently, sequentially or a combination thereof). For example, an LLM-based AI model can provide instructions for helping a user follow a recipe and the instructions can be based in part on another AI model that is derived from an ANN, a DNN, an RNN, etc. that is capable of discerning what part of the recipe the user is on (e.g., object and scene detection).

As AI training models evolve, the operations and experiences described herein could potentially be performed with different models other than those listed above, and a person skilled in the art would understand that the list above is non-limiting.

A user 602 can interact with an AI model through natural language inputs captured by a voice sensor, text inputs, or any other input modality that accepts natural language and/or a corresponding voice sensor module. In another instance, input is provided by tracking the eye gaze of a user 602 via a gaze tracker module. Additionally, the AI model can also receive inputs beyond those supplied by a user 602. For example, the AI can generate its response further based on environmental inputs (e.g., temperature data, image data, video data, ambient light data, audio data, GPS location data, inertial measurement (i.e., user motion) data, pattern recognition data, magnetometer data, depth data, pressure data, force data, neuromuscular data, heart rate data, temperature data, sleep data) captured in response to a user request by various types of sensors and/or their corresponding sensor modules. The sensors' data can be retrieved entirely from a single device (e.g., AR device 628) or from multiple devices that are in communication with each other (e.g., a system that includes at least two of an AR device 628, an MR device 632, the HIPD 642, the wrist-wearable device 626, etc.). The AI model can also access additional information (e.g., one or more servers 630, the computers 640, the mobile devices 650, and/or other electronic devices) via a network 625.

A non-limiting list of AI-enhanced functions includes but is not limited to image recognition, speech recognition (e.g., automatic speech recognition), text recognition (e.g., scene text recognition), pattern recognition, natural language processing and understanding, classification, regression, clustering, anomaly detection, sequence generation, content generation, and optimization. In some embodiments, AI-enhanced functions are fully or partially executed on cloud-computing platforms communicatively coupled to the user devices (e.g., the AR device 628, an MR device 632, the HIPD 642, the wrist-wearable device 626) via the one or more networks. The cloud-computing platforms provide scalable computing resources, distributed computing, managed AI services, interference acceleration, pre-trained models, APIs and/or other resources to support comprehensive computations required by the AI-enhanced function.

Example outputs stemming from the use of an AI model can include natural language responses, mathematical calculations, charts displaying information, audio, images, videos, texts, summaries of meetings, predictive operations based on environmental factors, classifications, pattern recognitions, recommendations, assessments, or other operations. In some embodiments, the generated outputs are stored on local memories of the user devices (e.g., the AR device 628, an MR device 632, the HIPD 642, the wrist-wearable device 626), storage options of the external devices (servers, computers, mobile devices, etc.), and/or storage options of the cloud-computing platforms.

The AI-based outputs can be presented across different modalities (e.g., audio-based, visual-based, haptic-based, and any combination thereof) and across different devices of the XR system described herein. Some visual-based outputs can include the displaying of information on XR augments of an XR headset, user interfaces displayed at a wrist-wearable device, laptop device, mobile device, etc. On devices with or without displays (e.g., HIPD 642), haptic feedback can provide information to the user 602. An AI model can also use the inputs described above to determine the appropriate modality and device(s) to present content to the user (e.g., a user walking on a busy road can be presented with an audio output instead of a visual output to avoid distracting the user 602).

### Example Augmented Reality Interaction

FIG. 6B shows the user 602 wearing the wrist-wearable device 626 and the AR device 628 and holding the HIPD 642. In the second AR system 600b, the wrist-wearable device 626, the AR device 628, and/or the HIPD 642 are used to receive and/or provide one or more messages to a contact of the user 602. In particular, the wrist-wearable device 626, the AR device 628, and/or the HIPD 642 detect and coordinate one or more user inputs to initiate a messaging application and prepare a response to a received message via the messaging application.

In some embodiments, the user 602 initiates, via a user input, an application on the wrist-wearable device 626, the AR device 628, and/or the HIPD 642 that causes the application to initiate on at least one device. For example, in the second AR system 600b the user 602 performs a hand gesture associated with a command for initiating a messaging application (represented by messaging user interface 612); the wrist-wearable device 626 detects the hand gesture; and, based on a determination that the user 602 is wearing the AR device 628, causes the AR device 628 to present a messaging user interface 612 of the messaging application. The AR device 628 can present the messaging user interface 612 to the user 602 via its display (e.g., as shown by user 602's field of view 610). In some embodiments, the application is initiated and can be run on the device (e.g., the wrist-wearable device 626, the AR device 628, and/or the HIPD 642) that detects the user input to initiate the application, and the device provides another device operational data to cause the presentation of the messaging application. For example, the wrist-wearable device 626 can detect the user input to initiate a messaging application, initiate and run the messaging application, and provide operational data to the AR device 628 and/or the HIPD 642 to cause presentation of the messaging application. Alternatively, the application can be initiated and run at a device other than the device that detected the user input. For example, the wrist-wearable device 626 can detect the hand gesture associated with initiating the messaging application and cause the HIPD 642 to run the messaging application and coordinate the presentation of the messaging application.

Further, the user 602 can provide a user input provided at the wrist-wearable device 626, the AR device 628, and/or the HIPD 642 to continue and/or complete an operation initiated at another device. For example, after initiating the messaging application via the wrist-wearable device 626 and while the AR device 628 presents the messaging user interface 612, the user 602 can provide an input at the HIPD 642 to prepare a response (e.g., shown by the swipe gesture performed on the HIPD 642). The user 602's gestures performed on the HIPD 642 can be provided and/or displayed on another device. For example, the user 602's swipe gestures performed on the HIPD 642 are displayed on a virtual keyboard of the messaging user interface 612 displayed by the AR device 628.

In some embodiments, the wrist-wearable device 626, the AR device 628, the HIPD 642, and/or other communicatively coupled devices can present one or more notifications to the user 602. The notification can be an indication of a new message, an incoming call, an application update, a status update, etc. The user 602 can select the notification via the wrist-wearable device 626, the AR device 628, or the HIPD 642 and cause presentation of an application or operation associated with the notification on at least one device. For example, the user 602 can receive a notification that a message was received at the wrist-wearable device 626, the AR device 628, the HIPD 642, and/or other communicatively coupled device and provide a user input at the wrist-wearable device 626, the AR device 628, and/or the HIPD 642 to review the notification, and the device detecting the user input can cause an application associated with the notification to be initiated and/or presented at the wrist-wearable device 626, the AR device 628, and/or the HIPD 642.

While the above example describes coordinated inputs used to interact with a messaging application, the skilled artisan will appreciate upon reading the descriptions that user inputs can be coordinated to interact with any number of applications including, but not limited to, gaming applications, social media applications, camera applications, web-based applications, financial applications, etc. For example, the AR device 628 can present to the user 602 game application data and the HIPD 642 can use a controller to provide inputs to the game. Similarly, the user 602 can use the wrist-wearable device 626 to initiate a camera of the AR device 628, and the user can use the wrist-wearable device 626, the AR device 628, and/or the HIPD 642 to manipulate the image capture (e.g., zoom in or out, apply filters) and capture image data.

While an AR device 628 is shown being capable of certain functions, it is understood that an AR device can be an AR device with varying functionalities based on costs and market demands. For example, an AR device may include a single output modality such as an audio output modality. In another example, the AR device may include a low-fidelity display as one of the output modalities, where simple information (e.g., text and/or low-fidelity images/video) is capable of being presented to the user. In yet another example, the AR device can be configured with face-facing light emitting diodes (LEDs) configured to provide a user with information, e.g., an LED around the right-side lens can illuminate to notify the wearer to turn right while directions are being provided or an LED on the left-side can illuminate to notify the wearer to turn left while directions are being provided. In another embodiment, the AR device can include an outward-facing projector such that information (e.g., text information, media) may be displayed on the palm of a user's hand or other suitable surface (e.g., a table, whiteboard). In yet another embodiment, information may also be provided by locally dimming portions of a lens to emphasize portions of the environment in which the user's attention should be directed. Some AR devices can present AR augments either monocularly or binocularly (e.g., an AR augment can be presented at only a single display associated with a single lens as opposed presenting an AR augmented at both lenses to produce a binocular image). In some instances an AR device capable of presenting AR augments binocularly can optionally display AR augments monocularly as well (e.g., for power-saving purposes or other presentation considerations). These examples are non-exhaustive and features of one AR device described above can be combined with features of another AR device described above. While features and experiences of an AR device have been described generally in the preceding sections, it is understood that the described functionalities and experiences can be applied in a similar manner to an MR headset, which is described below in the proceeding sections.

### Example Mixed Reality Interaction

Turning to FIGS. 6C-1 and 6C-2, the user 602 is shown wearing the wrist-wearable device 626 and an MR device 632 (e.g., a device capable of providing either an entirely VR experience or an MR experience that displays object(s) from a physical environment at a display of the device) and holding the HIPD 642. In the third MR system 600c, the wrist-wearable device 626, the MR device 632, and/or the HIPD 642 are used to interact within an MR environment, such as a VR game or other MR/VR application. While the MR device 632 presents a representation of a VR game (e.g., first MR game environment 620) to the user 602, the wrist-wearable device 626, the MR device 632, and/or the HIPD 642 detect and coordinate one or more user inputs to allow the user 602 to interact with the VR game.

In some embodiments, the user 602 can provide a user input via the wrist-wearable device 626, the MR device 632, and/or the HIPD 642 that causes an action in a corresponding MR environment. For example, the user 602 in the third MR system 600c (shown in FIG. 6C-1) raises the HIPD 642 to prepare for a swing in the first MR game environment 620. The MR device 632, responsive to the user 602 raising the HIPD 642, causes the MR representation of the user 622 to perform a similar action (e.g., raise a virtual object, such as a virtual sword 624). In some embodiments, each device uses respective sensor data and/or image data to detect the user input and provide an accurate representation of the user 602's motion. For example, image sensors (e.g., SLAM cameras or other cameras) of the HIPD 642 can be used to detect a position of the HIPD 642 relative to the user 602's body such that the virtual object can be positioned appropriately within the first MR game environment 620; sensor data from the wrist-wearable device 626 can be used to detect a velocity at which the user 602 raises the HIPD 642 such that the MR representation of the user 622 and the virtual sword 624 are synchronized with the user 602's movements; and image sensors of the MR device 632 can be used to represent the user 602's body, boundary conditions, or real-world objects within the first MR game environment 620.

In FIG. 6C-2, the user 602 performs a downward swing while holding the HIPD 642. The user 602's downward swing is detected by the wrist-wearable device 626, the MR device 632, and/or the HIPD 642 and a corresponding action is performed in the first MR game environment 620. In some embodiments, the data captured by each device is used to improve the user's experience within the MR environment. For example, sensor data of the wrist-wearable device 626 can be used to determine a speed and/or force at which the downward swing is performed and image sensors of the HIPD 642 and/or the MR device 632 can be used to determine a location of the swing and how it should be represented in the first MR game environment 620, which, in turn, can be used as inputs for the MR environment (e.g., game mechanics, which can use detected speed, force, locations, and/or aspects of the user 602's actions to classify a user's inputs (e.g., user performs a light strike, hard strike, critical strike, glancing strike, miss) or calculate an output (e.g., amount of damage)).

FIG. 6C-2 further illustrates that a portion of the physical environment is reconstructed and displayed at a display of the MR device 632 while the MR game environment 620 is being displayed. In this instance, a reconstruction of the physical environment 646 is displayed in place of a portion of the MR game environment 620 when object(s) in the physical environment are potentially in the path of the user (e.g., a collision with the user and an object in the physical environment are likely). Thus, this example MR game environment 620 includes (i) an immersive VR portion 648 (e.g., an environment that does not have a corollary counterpart in a nearby physical environment) and (ii) a reconstruction of the physical environment 646 (e.g., table 629 and cup). While the example shown here is an MR environment that shows a reconstruction of the physical environment to avoid collisions, other uses of reconstructions of the physical environment can be used, such as defining features of the virtual environment based on the surrounding physical environment (e.g., a virtual column can be placed based on an object in the surrounding physical environment (e.g., a tree)).

While the wrist-wearable device 626, the MR device 632, and/or the HIPD 642 are described as detecting user inputs, in some embodiments, user inputs are detected at a single device (with the single device being responsible for distributing signals to the other devices for performing the user input). For example, the HIPD 642 can operate an application for generating the first MR game environment 620 and provide the MR device 632 with corresponding data for causing the presentation of the first MR game environment 620, as well as detect the user 602's movements (while holding the HIPD 642) to cause the performance of corresponding actions within the first MR game environment 620. Additionally or alternatively, in some embodiments, operational data (e.g., sensor data, image data, application data, device data, and/or other data) of one or more devices is provided to a single device (e.g., the HIPD 642) to process the operational data and cause respective devices to perform an action associated with processed operational data.

In some embodiments, the user 602 can wear a wrist-wearable device 626, wear an MR device 632, wear smart textile-based garments 638 (e.g., wearable haptic gloves), and/or hold an HIPD 642 device. In this embodiment, the wrist-wearable device 626, the MR device 632, and/or the smart textile-based garments 638 are used to interact within an MR environment (e.g., any AR or MR system described above in reference to FIGS. 6A-6B). While the MR device 632 presents a representation of an MR game (e.g., second MR game environment 620) to the user 602, the wrist-wearable device 626, the MR device 632, and/or the smart textile-based garments 638 detect and coordinate one or more user inputs to allow the user 602 to interact with the MR environment.

In some embodiments, the user 602 can provide a user input via the wrist-wearable device 626, an HIPD 642, the MR device 632, and/or the smart textile-based garments 638 that causes an action in a corresponding MR environment. In some embodiments, each device uses respective sensor data and/or image data to detect the user input and provide an accurate representation of the user 602's motion. While four different input devices are shown (e.g., a wrist-wearable device 626, an MR device 632, an HIPD 642, and a smart textile-based garment 638) each one of these input devices entirely on its own can provide inputs for fully interacting with the MR environment. For example, the wrist-wearable device can provide sufficient inputs on its own for interacting with the MR environment. In some embodiments, if multiple input devices are used (e.g., a wrist-wearable device and the smart textile-based garment 638) sensor fusion can be utilized to ensure inputs are correct. While multiple input devices are described, it is understood that other input devices can be used in conjunction or on their own instead, such as but not limited to external motion-tracking cameras, other wearable devices fitted to different parts of a user, apparatuses that allow for a user to experience walking in an MR environment while remaining substantially stationary in the physical environment, etc.

As described above, the data captured by each device is used to improve the user's experience within the MR environment. Although not shown, the smart textile-based garments 638 can be used in conjunction with an MR device and/or an HIPD 642.

While some experiences are described as occurring on an AR device and other experiences are described as occurring on an MR device, one skilled in the art would appreciate that experiences can be ported over from an MR device to an AR device, and vice versa.

### Other Interactions

While numerous examples are described in this application related to extended-reality environments, one skilled in the art would appreciate that certain interactions may be possible with other devices. For example, a user may interact with a robot (e.g., a humanoid robot, a task specific robot, or other type of robot) to perform tasks inclusive of, leading to, and/or otherwise related to the tasks described herein. In some embodiments, these tasks can be user specific and learned by the robot based on training data supplied by the user and/or from the user's wearable devices (including head-worn and wrist-worn, among others) in accordance with techniques described herein. As one example, this training data can be received from the numerous devices described in this application (e.g., from sensor data and user-specific interactions with head-wearable devices, wrist-wearable devices, intermediary processing devices, or any combination thereof). Other data sources are also conceived outside of the devices described here. For example, AI models for use in a robot can be trained using a blend of user-specific data and non-user specific-aggregate data. The robots may also be able to perform tasks wholly unrelated to extended reality environments, and can be used for performing quality-of-life tasks (e.g., performing chores, completing repetitive operations, etc.). In certain embodiments or circumstances, the techniques and/or devices described herein can be integrated with and/or otherwise performed by the robot.

Some definitions of devices and components that can be included in some or all of the example devices discussed are defined here for ease of reference. A skilled artisan will appreciate that certain types of the components described may be more suitable for a particular set of devices, and less suitable for a different set of devices. But subsequent reference to the components defined here should be considered to be encompassed by the definitions provided.

In some embodiments example devices and systems, including electronic devices and systems, will be discussed. Such example devices and systems are not intended to be limiting, and one of skill in the art will understand that alternative devices and systems to the example devices and systems described herein may be used to perform the operations and construct the systems and devices that are described herein.

As described herein, an electronic device is a device that uses electrical energy to perform a specific function. It can be any physical object that contains electronic components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronic components.

The foregoing descriptions of FIGS. 6A-6C-2 provided above are intended to augment the description provided in reference to FIGS. 1A-5. While terms in the following description may not be identical to terms used in the foregoing description, a person having ordinary skill in the art would understand these terms to have the same meaning.

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt in or opt out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A device comprising:
a wearable structure configured to couple to a body of a user, the wearable structure comprising:
a light emitter coupled to a plurality of fiber optic components and configured to transmit a light output to the plurality of fiber optic components;
an optical switch coupled to the plurality of fiber optic components and having a plurality of inputs and one or more outputs;
a photo sensor coupled to at least one output of the one or more outputs of the optical switch, wherein the photo sensor is configured to detect a reflected portion of the light output from the light emitter and output a corresponding photo sensor signal;
control circuitry coupled to the photo sensor and configured to:
determine a curvature of a respective fiber optic component of the plurality of fiber optic components based on analysis of the corresponding photo sensor signal; and
determine a pose of the user based on the curvature of the respective fiber optic component; and
a power source configured to provide power to the control circuitry, the light emitter, the optical switch, and the photo sensor; and
the plurality of fiber optic components, each fiber optic component of the plurality of fiber optic components comprising a respective set of passive sensors, each passive sensor of the respective set of passive sensors configured to reflect a portion of light transmitted to the fiber optic component.

2. The device of claim 1, wherein the respective set of passive sensors comprises a set of fiber Bragg grating (FBG) sensors.

3. The device of claim 1, wherein each passive sensor of the respective set of passive sensors is configured to reflect a respective predetermined wavelength.

4. The device of claim 1, wherein the respective set of passive sensors is arranged in a meandering pattern extending from the wearable structure toward an end of a respective limb of the user.

5. The device of claim 1, wherein the respective set of passive sensors is arranged in a linear pattern extending from the wearable structure toward an end of a respective limb of the user.

6. The device of claim 1, wherein each fiber optic component of the plurality of fiber optic components is arranged to extend from the wearable structure along a different part of the body of the user.

7. The device of claim 1, wherein the device comprises a wearable glove, a wrist-wearable device, or a belt.

8. The device of claim 1, wherein the plurality of fiber optic components are embedded in a material of the device.

9. The device of claim 1, wherein the control circuitry is configured to receive one or more additional photo sensor signals from one or more photo sensors that are not components of the wearable structure, wherein the pose of the user is further based on the one or more additional photo sensor signals.

10. The device of claim 1, wherein the light emitter, the optical switch, and the photo sensor are components of a photonic integrated circuit.

11. The device of claim 1, wherein the light emitter comprises a laser component.

12. A method of pose estimation, comprising:
transmitting a light output from a light emitter to a plurality of fiber optic components;
detecting, via a photo sensor, a reflected portion of the light output, wherein the reflected portion is reflected by one or more passive sensors within the plurality of fiber optic components;
generating, via the photo sensor, a photo sensor signal based on the reflected portion of the light output;
determining a curvature for the plurality of fiber optic components based on the photo sensor signal; and
determining a pose of a user based on the curvature of the plurality of fiber optic components.

13. The method of claim 12, wherein the curvature for the plurality of fiber optic components is determined by a processor of a wearable device that comprises the light emitter, the photo sensor, and the plurality of fiber optic components.

14. The method of claim 12, wherein the pose of the user based is determined by a processor of a wearable device that comprises the light emitter, the photo sensor, and the plurality of fiber optic components.

15. The method of claim 12, wherein the curvature for the plurality of fiber optic components is determined based on a plurality of photo sensor signals, each photo sensor signal of the plurality of photo sensor signals corresponding to a different passive sensor within the plurality of fiber optic components.

16. The method of claim 12, wherein the pose of the user is determined by mapping the curvature to human skeletal data.

17. A non-transitory computer-readable storage medium storing instructions that, when executed by control circuitry of a wearable device, cause the wearable device to:
transmit a light output from a light emitter to a plurality of fiber optic components;
detect, via a photo sensor, a reflected portion of the light output, wherein the reflected portion is reflected by one or more passive sensors within the plurality of fiber optic components;
generate, via the photo sensor, a photo sensor signal based on the reflected portion of the light output;
determine a curvature for the plurality of fiber optic components based on the photo sensor signal; and
determine a pose of a user based on the curvature of the plurality of fiber optic components.

18. The non-transitory computer-readable storage medium of claim 17, wherein the pose of the user is determined by mapping the curvature to human skeletal data.

19. The non-transitory computer-readable storage medium of claim 17, wherein the pose of the user is determined using a kinematics algorithm.

20. The non-transitory computer-readable storage medium of claim 17, wherein the light emitter and the photo sensor are components of a photonic integrated circuit.
